(19) United States
(12) Patent Application Publication
(10) Pub. No.: US 2025/0049653 A1
(43) Pub. Date: Feb. 13, 2025

(54) NON-STICKY SMOOTH STABLE COMPOSITION

(71) Applicant: L'OREAL, Paris (FR)

(72) Inventors: Kazuhiko Maruyama, Kawasaki-shi (JP); Tomoko Mizuno, Kawasaki-shi (JP)

(21) Appl. No.: 18/717,319

(22) PCT Filed: Dec. 9, 2022
(86) PCT No.: PCT/JP2022/046300

§ 371 Date: Jun. 6, 2024

(30) Foreign Application Priority Data
Dec. 17, 2021 (JP) 2021-205211
Jan. 19, 2022 (FR) 2200445

(51) Int. Cl. A61K 8/06; A61Q 19/08

(52) U.S. Cl. CPC A61K 8/062; A61Q 19/08; A61K 2800/28; A61K 2800/592; A61K 2800/596

(57) ABSTRACT

An O/W emulsion contains: (a) at least one oil; (b) at least one fatty alcohol; (c) at least one acid selected from hydroxyl acids, phytic acid, and mixtures thereof; (d) at least one polyglyceryl fatty acid ester having a C₆-C₃₂ fatty acid residue; (e) at least one thickener; (f) at least one powder; and (g) water, wherein the HLB of the (d) polyglyceryl fatty acid ester having a C₆-C₃₂ fatty acid residue or the weighted average of HLB values of the (d) polyglyceryl fatty acid esters having a C₆-C₃₂ fatty acid residue is between 8.0 and 11.5. The composition includes the (c) acid, but it is stable and can provide no sticky feeling or a reduced sticky feeling as well as a non-greasy feeling or smooth feeling.

### TECHNICAL FIELD

The present invention relates to a composition, preferably a cosmetic or dermatological composition, which is non-sticky, smooth and stable.

### BACKGROUND ART

Peeling is a well-known means for improving the appearance of the surface of the skin, in particular for treating visible and/or tactile irregularities of the human skin, and for example to attenuate defects of pigmentation such as skin freckles or the marks due to acne or varicella, or to smooth irregularities in the texture of the skin, in particular wrinkles or minor wrinkles.

This peeling has the effect of removing part of the skin to be treated (epidermis and possibly upper layer of dermis) by chemical methods such as the application of compositions containing a peeling agent(s) stimulating the desquamation of the skin, for example alpha-hydroxy acids (AHAs) such as glycolic acid or beta-hydroxy acids (BHAs) such as salicylic acid, and phytic acid, or else other active substances such as retinoic acid, resorcinol, trichloroacetic acid or phenol.

### DISCLOSURE OF INVENTION

It has been found that, if a peeling agent is incorporated into an emulsion such as a milky lotion, this incorporation often provides a sticky feeling to the touch or a greasy feeling to the touch and tends to be unstable, in particular unstable over time and/or under temperature changes.

An objective of the present invention is to provide a composition which provides no sticky feeling or a reduced sticky feeling after application, as well as a non-greasy feeling or smooth feeling to the touch, and which is stable, in particular stable over time and/or under temperature changes, even if the composition includes a peeling agent.

The above objective of the present invention can be achieved by a composition, in the form of an O/W emulsion, comprising:
(a) at least one oil;
(b) at least one fatty alcohol;
(c) at least one acid selected from hydroxyl acids, phytic acid, and mixtures thereof;
(d) at least one polyglyceryl fatty acid ester having a C₆-C₃₂ fatty acid residue;
(e) at least one thickener;
(f) at least one powder; and
(g) water,
wherein
the HLB of the (d) polyglyceryl fatty acid ester having a C₆-C₃₂ fatty acid residue or the weighted average of HLB values of the (d) polyglyceryl fatty acid esters having a C₆-C₃₂ fatty acid residue is between 8.0 and 11.5.

The (a) oil may be selected from the group consisting of ester oils, triglyceride oils and mixtures thereof.

The amount of the (a) oil in the composition according to the present invention may range from 0.01% to 30% by weight, preferably from 0.1% to 25% by weight, and more preferably from 1% to 20% by weight, relative to the total weight of the composition.

The (b) fatty alcohol may be selected from the group consisting of cetyl alcohol, cetearyl alcohol, stearyl alcohol, behenyl alcohol, oleyl alcohol, and mixtures thereof.

The amount of the (b) fatty alcohol in the composition according to the present invention may range from 0.01% to 15% by weight, preferably from 0.05% to 10% by weight, and more preferably from 0.1% to 5% by weight, relative to the total weight of the composition.

The above hydroxyl acid for the (c) acid may be selected from alpha-hydroxy acids, beta-hydroxy acids, and mixtures thereof.

The amount of the (c) acid in the composition according to the present invention may range from 0.01% to 15% by weight, preferably from 0.1% to 10% by weight, and more preferably from 1% to 8% by weight, relative to the total weight of the composition.

The (d) polyglyceryl fatty acid ester having a C₆-C₃₂ fatty acid residue may have a polyglyceryl moiety derived from 2 to 10 glycerins, preferably 2 to 8 glycerins, and more preferably from 2 to 6 glycerins.

The amount of the (d) polyglyceryl fatty acid ester having a C₆-C₃₂ fatty acid residue in the composition according to the present invention may range from 0.01% to 20% by weight, preferably from 0.05% to 15% by weight, and more preferably from 0.1% to 10% by weight, relative to the total weight of the composition.

The (e) thickener may be selected from polysaccharides, AMPS (co)polymers, and a mixture thereof.

The amount of the (e) thickener in the composition according to the present invention may range from 0.01% to 10% by weight, preferably from 0.05% to 5% by weight, and more preferably from 0.1% to 1% by weight, relative to the total weight of the composition.

The (f) powder may be selected from the group consisting of pigments, fillers and mixtures thereof.

The amount of the (f) powder(s) in the composition according to the present invention may range from 0.1% to 15% by weight, preferably from 0.5% to 10% by weight, and more preferably from 1% to 5% by weight, relative to the total weight of the composition.

The composition according to the present invention may further comprise (h) at least one compound selected from Vitamin B3 and derivatives thereof.

The present invention also relates to a cosmetic process for treating a keratin substance, comprising the step of applying the composition according to the present invention to the keratin substance.

### BEST MODE FOR CARRYING OUT THE INVENTION

After diligent research, the inventors have discovered a new approach to provide a composition which provides no sticky feeling or a reduced sticky feeling after application, as well as a non-greasy feeling or a smooth feeling to the touch, and which is stable, in particular stable over time and/or under temperature changes, even if the composition includes a peeling agent, by using a specific combination of selected ingredients under certain conditions.

Thus, one aspect of the present invention is a composition comprising:
(a) at least one oil;
(b) at least one fatty alcohol;
(c) at least one acid selected from hydroxyl acids, phytic acid, and mixtures thereof;
(d) at least one polyglyceryl fatty acid ester having a C₆-C₃₂ fatty acid residue;
(e) at least one thickener;
(f) at least one powder; and
(g) water,
wherein
the HLB of the (d) polyglyceryl fatty acid ester having a C₆-C₃₂ fatty acid residue or the weighted average of HLB values of the (d) polyglyceryl fatty acid esters having a C₆-C₃₂ fatty acid residue is between 8.0 and 11.5.

The composition according to the present invention includes a peeling agent, i.e., the (c) acid, but can provide no sticky feeling or a reduced sticky feeling as well as a non-greasy feeling or a smooth feeling to the touch.

Therefore, the composition according to the present invention can provide an excellent feeling during use, in particular on feeling of the skin during and after application of the composition.

The term “sticky” here means a property which provides a tacky feeling to the skin.

The term “greasy” here means a property which provides a slimy feeling to the skin.

The composition according to the present invention is stable just after the preparation of the composition and a long time after the preparation of the composition, even under temperature changes from cold to hot temperature. Therefore, the composition according to the present invention is stable over time, and can be stored for a long period of time even under temperature changes occurring from winter to summer.

If the composition according to the present invention includes (h) at least one compound selected from Vitamin B3 and derivatives thereof, the composition according to the present invention can provide more excellent cosmetic effects in terms of skin tone improvements.

Hereinafter, the composition according to the present invention will be explained in a more detailed manner.

### [Oil]

The composition according to the present invention comprises (a) at least one oil. A single type of oil may be used, but two or more different types of oils may be used in combination.

Here, “oil” means a fatty compound or substance which is in the form of a liquid or a paste or a solid at room temperature (25° C.) under atmospheric pressure (760 mmHg). As the (a) oil(s), those generally used in cosmetics can be used alone or in combination thereof. These oils may be volatile or non-volatile.

The (a) oil may be a non-polar oil such as a hydrocarbon oil, a silicone oil, or the like; a polar oil such as a plant or animal oil and an ester oil or an ether oil; or a mixture thereof.

The (a) oil may be selected from the group consisting of oils of plant or animal origin, synthetic oils, silicone oils, and hydrocarbon oils.

As examples of plant oils, mention may be made of, for example, linseed oil, camellia oil, macadamia nut oil, corn oil, mink oil, olive oil, avocado oil, sasanqua oil, castor oil, safflower oil, jojoba oil, jojoba esters, sunflower oil, almond oil, rapeseed oil, sesame oil, soybean oil, peanut oil, and mixtures thereof.

As examples of synthetic oils, mention may be made of alkane oils such as isododecane and isohexadecane, ester oils, ether oils, and artificial triglycerides.

The ester oils are preferably liquid esters of saturated or unsaturated, linear or branched C₁-C₂₆ aliphatic monoacids or polyacids and of saturated or unsaturated, linear or branched C₁-C₂₆ aliphatic monoalcohols or polyalcohols, the total number of carbon atoms of the esters being greater than or equal to 10.

Preferably, for the esters of monoalcohols, at least one from among the alcohol and the acid from which the esters of the present invention are derived is branched.

Among the monoesters of monoacids and of monoalcohols, mention may be made of ethyl palmitate, ethyl hexyl palmitate, isopropyl palmitate, dicaprylyl carbonate, alkyl myristates such as isopropyl myristate or ethyl myristate, cetyl palmitate, isocetyl stearate, 2-ethylhexyl isononanoate, isononyl isononanoate, isodecyl neopentanoate and isostearyl neopentanoate.

Esters of C₄-C₂₂ dicarboxylic or tricarboxylic acids and of C₁-C₂₂ alcohols, and esters of monocarboxylic, dicarboxylic or tricarboxylic acids and of non-sugar C₄-C₂₆ dihydroxy, trihydroxy, tetrahydroxy or pentahydroxy alcohols may also be used.

Mention may especially be made of: diethyl sebacate; isopropyl lauroyl sarcosinate; diisopropyl sebacate; bis(2-ethylhexyl) sebacate; diisopropyl adipate; di-n-propyl adipate; dioctyl adipate; bis(2-ethylhexyl) adipate; diisostearyl adipate; bis(2-ethylhexyl) maleate; triisopropyl citrate; triisocetyl citrate; triisostearyl citrate; glyceryl trilactate; glyceryl trioctanoate; trioctyldodecyl citrate; trioleyl citrate; neopentyl glycol diheptanoate; diethylene glycol diisononanoate.

As ester oils, one can use sugar esters and diesters of C₆-C₃₀ and preferably C₁₂-C₂₂ fatty acids. It is recalled that the term “sugar” means oxygen-bearing hydrocarbon-based compounds containing several alcohol functions, with or without aldehyde or ketone functions, and which comprise at least 4 carbon atoms. These sugars may be monosaccharides, oligosaccharides or polysaccharides.

Examples of suitable sugars that may be mentioned include sucrose (or saccharose), glucose, galactose, ribose, fucose, maltose, fructose, mannose, arabinose, xylose and lactose, and derivatives thereof, especially alkyl derivatives, such as methyl derivatives, for instance methylglucose.

The sugar esters of fatty acids may be chosen especially from the group comprising the esters or mixtures of esters of sugars described previously and of linear or branched, saturated or unsaturated C₆-C₃₀ and preferably C₁₂-C₂₂ fatty acids. If they are unsaturated, these compounds may have one to three conjugated or non-conjugated carbon-carbon double bonds.

The esters according to this variant may also be selected from monoesters, diesters, triesters, tetraesters and polyesters, and mixtures thereof.

These esters may be, for example, oleates, laurates, palmitates, myristates, behenates, cocoates, stearates, linoleates, linolenates, caprates and arachidonates, or mixtures thereof such as, especially, oleopalmitate, oleostearate and palmitostearate mixed esters, as well as pentaerythrityl tetraethyl hexanoate.

More particularly, use is made of monoesters and diesters and especially sucrose, glucose or methylglucose monooleates or dioleates, stearates, behenates, oleopalmitates, linoleates, linolenates and oleostearates.

An example that may be mentioned is the product sold under the name Glucate® DO by the company Amerchol, which is a methylglucose dioleate.

As examples of preferable ester oils, mention may be made of, for example, diisopropyl adipate, dioctyl adipate, 2-ethylhexyl hexanoate, ethyl laurate, cetyl octanoate, octyldodecyl octanoate, isodecyl neopentanoate, myristyl propionate, 2-ethylhexyl 2-ethylhexanoate, 2-ethylhexyl octanoate, 2-ethylhexyl caprylate/caprate, methyl palmitate, ethyl palmitate, isopropyl palmitate, dicaprylyl carbonate, isopropyl lauroyl sarcosinate, isononyl isononanoate, ethylhexyl palmitate, isohexyl laurate, hexyl laurate, isocetyl stearate, isopropyl isostearate, isopropyl myristate, isodecyl oleate, glyceryl tri(2-ethylhexanoate), pentaerythrithyl tetra(2-ethylhexanoate), 2-ethylhexyl succinate, diethyl sebacate, and mixtures thereof.

As examples of artificial triglycerides, mention may be made of, for example, capryl caprylyl glycerides, glyceryl trimyristate, glyceryl tripalmitate, glyceryl trilinolenate, glyceryl trilaurate, glyceryl tricaprate, glyceryl tricaprylate, glyceryl tri(caprate/caprylate) and glyceryl tri(caprate/caprylate/linolenate).

As examples of silicone oils, mention may be made of, for example, linear organopolysiloxanes such as dimethylpolysiloxane, methylphenylpolysiloxane, methylhydrogenpolysiloxane, and the like; cyclic organopolysiloxanes such as cyclohexasiloxane, octamethylcyclotetrasiloxane, decamethylcyclopentasiloxane, dodecamethylcyclohexasiloxane, and the like; and mixtures thereof.

Preferably, the silicone oils are chosen from liquid polydialkylsiloxanes, especially liquid polydimethylsiloxanes (PDMS) and liquid polyorganosiloxanes comprising at least one aryl group.

These silicone oils may also be organomodified. The organomodified silicones that can be used in accordance with the present invention are silicone oils as defined above and comprise in their structure one or more organofunctional groups attached via a hydrocarbon-based group.

Organopolysiloxanes are defined in greater detail in Walter Noll's Chemistry and Technology of Silicones (1968), Academic Press. They may be volatile or non-volatile.

When they are volatile, the silicones are more particularly chosen from those having a boiling point of between 60° C. and 260° C., and even more particularly from:
(i) cyclic polydialkylsiloxanes comprising from 3 to 7 and preferably 4 to 5 silicon atoms. These are, for example, octamethylcyclotetrasiloxane sold in particular under the name Volatile Silicone® 7207 by Union Carbide or Silbione® 70045 V2 by Rhodia, decamethylcyclopentasiloxane sold under the name Volatile Silicone® 7158 by Union Carbide, Silbione® 70045 V5 by Rhodia, and dodecamethylcyclopentasiloxane sold under the name Silsoft 1217 by Momentive Performance Materials, and mixtures thereof. Mention may also be made of cyclocopolymers of the type such as dimethylsiloxane/methylalkylsiloxane, such as Silicone Volatile® FZ 3109 sold by the company Union Carbide, of formula:

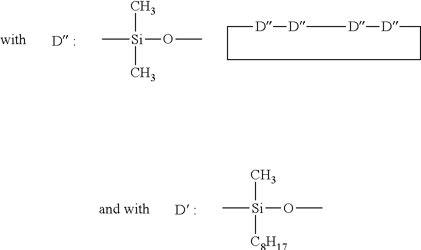

Mention may also be made of mixtures of cyclic polydialkylsiloxanes with organosilicon compounds, such as the mixture of octamethylcyclotetrasiloxane and tetratrimethylsilylpentaerythritol (50/50) and the mixture of octamethylcyclotetrasiloxane and oxy-1,1′-bis(2,2,2′,2′,3,3′-hexatrimethylsilyloxy)neopentane;
(ii) linear volatile polydialkylsiloxanes containing 2 to 9 silicon atoms and having a viscosity of less than or equal to 5×10⁻⁶ m²/s at 25° C. An example is decamethyltetrasiloxane sold in particular under the name SH 200 by the company Toray Silicone. Silicones belonging to this category are also described in the article published in Cosmetics and Toiletries, Vol. 91, January 76, pp. 27-32, Todd & Byers, Volatile Silicone Fluids for Cosmetics. The viscosity of the silicones is measured at 25° C. according to ASTM standard 445 Appendix C.

Non-volatile polydialkylsiloxanes may also be used. These non-volatile silicones are more particularly chosen from polydialkylsiloxanes, among which mention may be made mainly of polydimethylsiloxanes containing trimethylsilyl end groups.

Among these polydialkylsiloxanes, mention may be made, in a non-limiting manner, of the following commercial products:
the Silbione® oils of the 47 and 70 047 series or the Mirasil® oils sold by Rhodia, for instance the oil 70 047 V 500 000;
the oils of the Mirasil® series sold by the company Rhodia;
the oils of the 200 series from the company Dow Corning, such as DC200 with a viscosity of 60 000 mm²/s;
the Viscasil® oils from General Electric and certain oils of the SF series (SF 96, SF 18) from General Electric.

Mention may also be made of polydimethylsiloxanes containing dimethylsilanol end groups known under the name dimethiconol (CTFA), such as the oils of the 48 series from the company Rhodia.

Among the silicones containing aryl groups are polydiarylsiloxanes, especially polydiphenylsiloxanes and polyalkylarylsiloxanes. Examples that may be mentioned include the products sold under the following names:
the Silbione® oils of the 70 641 series from Rhodia;
the oils of the Rhodorsil® 70 633 and 763 series from Rhodia;
the oil Dow Corning 556 Cosmetic Grade Fluid from Dow Corning;
the silicones of the PK series from Bayer, such as the product PK20;
certain oils of the SF series from General Electric, such as SF 1023, SF 1154, SF 1250 and SF 1265.

The organomodified liquid silicones may especially contain polyethyleneoxy and/or polypropyleneoxy groups. Mention may thus be made of the silicone KF-6017 proposed by Shin-Etsu, and the oils Silwet® L722 and L77 from the company Union Carbide.

Hydrocarbon oils may be chosen from:
linear or branched, optionally cyclic, C₆-C₁₆ lower alkanes. Examples that may be mentioned include hexane, undecane, dodecane, tridecane, and isoparaffins, for instance isohexadecane, isododecane and isodecane; and
linear or branched hydrocarbons containing more than 16 carbon atoms, such as liquid paraffins, liquid petroleum jelly, polydecenes and hydrogenated polyisobutenes such as Parleam®, and squalane.

As preferable examples of hydrocarbon oils, mention may be made of, for example, linear or branched hydrocarbons such as isohexadecane, isododecane, squalane, mineral oil (e.g., liquid paraffin), paraffin, vaseline or petrolatum, naphthalenes, and the like; hydrogenated polyisobutene, isoeicosan, and decene/butene copolymer; and mixtures thereof.

In one embodiment, the (a) oil may be chosen from triglyceride oils.

The triglyceride oil comprises at least one triglyceride. The triglyceride may be referred to as a triacyl glycerol, and three fatty acids or two fatty acids and one non-fatty acid, and one glycerol are esterified in the triglyceride.

The fatty acid may have, for example, 4 or more, 6 or more, 8 or more, or 10 or more carbon atoms, and 30 or fewer, 28 or fewer, 26 or fewer, or 24 or fewer carbon atoms. The fatty acid may have a different carbon chain length of, for example, from 4 to 30 carbon atoms, preferably from 6 to 28 carbon atoms, more preferably from 8 to 26 carbon atoms, and even more preferably from 10 to 24 carbon atoms. The carbon chain may be linear or branched.

The fatty acid may be saturated or unsaturated.

As examples of the saturated fatty acid, mention may be made of, for example, palmitic acid, heptadecanoic acid, stearic acid, nonadecanoic acid, arachidic acid, behenic acid, tetradocosanoic acid, hexadocosanoic acid, and octadocosanoic acid.

As examples of the unsaturated fatty acid, mention may be made of, for example, palmitoleic acid, oleic acid, elaidic acid, vaccenic acid, gadoleic acid, eicosenoic acid, erucic acid, nervonic acid, linoleic acid, eicosadienoic acid, docosadienoic acid, linolenic acid, pinolenic acid, eleostearic acid, mead acid, eicosatrienoic acid, stearidonic acid, arachidonic acid, eicosatetraenoic acid, adrenic acid, bosseopentaenoic acid, eicosapentaenoic acid, osbond acid, clupanodonic acid, tetracosapentaenoic acid, docosahexaenoic acid, and nisinic acid.

Examples of the non-fatty acid may be dicarboxylic acids which may have, for example, 1 or more, 2 or more, 3 or more, or 4 or more carbon atoms, and 12 or fewer, 10 or fewer, 8 or fewer, or 6 or fewer carbon atoms. The non-fatty acid may have a different carbon chain length of, for 50 example, from 1 to 12 carbon atoms, preferably from 2 to 10 carbon atoms, more preferably from 3 to 8 carbon atoms, and even more preferably from 4 to 6 carbon atoms. The carbon chain may be linear or branched.

The non-fatty acid, preferably dicarboxylic acid, may be saturated or unsaturated.

As examples of the saturated non-fatty acid, preferably saturated dicarboxylic acid, mention may be made of, for example, oxalic acid, malonic acid, succinic acid, glutaric acid, adipic acid, pimelic acid, suberic acid, azelaic acid and sebacic acid.

As examples of the unsaturated fatty acid, mention may be made of, for example, maleic acid, fumaric acid, citraconic acid, mesaconic acid and 2-pentenoic acid.

The triglyceride oil which may be suitable for the present invention is of plant origin. In other words, it is preferable that the triglyceride oil be selected from plant oils.

The plant oil may be selected from plant-extracted oils, plant-extracted butters, and mixtures thereof.

Among the plant-extracted oils, the following may be cited: jojoba oil, babassu oil, sunflower oil, olive oil, canola oil, coconut oil, meadowfoam seed oil; Brazil nut oil, marula oil, maize oil, argan oil, soybean oil, marrow oil, grapeseed oil, linseed oil, sesame oil, hazelnut oil, apricot oil, macadamia oil, arara oil, coriander oil, castor oil, avocado oil, shea butter oil, rapeseed oil, and copra oil.

Among the plant-extracted butters, the following may be cited: shea butter, Nilotica shea butter (Butyrospermum parkii), galam butter (Butyrospermum parkii), Borneo butter or fat or tengkawang tallow (Shorea stenoptera), shorea butter, illipe butter, madhuca butter or (Bassia) Madhuca longifolia butter, mowrah butter (Madhuca latifolia), katiau butter (Madhuca mottleyana), phulwara butter (M. butyracea), mango butter (Mangifera indica), murumuru butter (Astrocaryum murumuru), kokum butter (Garcinia indica), ucuuba butter (Virola sebifera), tucuma butter, painya (kpangnan) butter (Pentadesma butyracea), coffee butter (Coffea arabica), apricot butter (Prunus armeniaca), macadamia butter (Macadamia ternifolia), grapeseed butter (Vitis vinifera), avocado butter (Persea gratissima), olive butter (Olea europaea), sweet almond butter (Prunus amygdalus dulcis), cocoa butter (Theobroma cacao), and sunflower butter.

In a preferable embodiment, the triglyceride oil may be selected from caprylic/capric/succinic triglyceride, shea butter, and a mixture thereof.

It is preferable that the (a) oil be chosen from ester oils, triglyceride oils, hydrocarbon oils, silicone oils, and mixtures thereof, and more preferably chosen from ester oils, triglyceride oils and mixtures thereof.

It is preferable that the (a) oil be chosen from oils with a molecular weight below 600 g/mol.

The amount of the (a) oil(s) in the composition according to the present invention may be 0.01% by weight or more, preferably 0.1% by weight or more, and more preferably 1% by weight or more, relative to the total weight of the composition.

On the other hand, the amount of the (a) oil(s) in the composition according to the invention may be 30% by weight or less, preferably 25% by weight or less, and more preferably 20% by weight or less, relative to the total weight of the composition.

The amount of the (a) oil(s) in the composition according to the present invention may range from 0.01% to 30% by weight, preferably from 0.1% to 25% by weight, more preferably from 1% to 20% by weight, relative to the total weight of the composition.

### [Fatty Alcohol]

The composition according to the present invention comprises (b) at least one fatty alcohol. A single type of fatty alcohol may be used, but two or more different types of fatty alcohol may be used in combination.

The term “fatty” here means the inclusion of a relatively large number of carbon atoms. Thus, alcohols which have 6 or more, preferably 8 or more, and more preferably 10 or more carbon atoms are encompassed within the scope of fatty alcohols. The fatty alcohols may be saturated or unsaturated. The fatty alcohol may be linear or branched. Two or more fatty alcohols may be used in combination.

The (b) fatty alcohol may have the structure R—OH wherein R is chosen from saturated and unsaturated, linear and branched radicals containing from 8 to 40 carbon atoms, for example from 8 to 30 carbon atoms. In at least one embodiment, R is chosen from C₁₂-C₂₄ alkyl and C₁₂-C₂₄ alkenyl groups. R may be or may not be substituted with at least one hydroxyl group.

Non-limiting examples of the (b) fatty alcohols that may be mentioned include lauryl alcohol, cetyl alcohol, stearyl alcohol, oleyl alcohol, behenyl alcohol, linoleyl alcohol, undecylenyl alcohol, palmitoleyl alcohol, arachidonyl alcohol, erucyl alcohol, cetearyl alcohol, and a mixture thereof.

Examples of suitable fatty alcohols include, but are not limited to, cetyl alcohol, cetearyl alcohol, stearyl alcohol, behenyl alcohol, oleyl alcohol, and a mixture thereof.

The fatty alcohol may represent a mixture of fatty alcohols, which means that several species of fatty alcohol may coexist, in the form of a mixture, in a commercial product.

According to at least one embodiment, the fatty alcohol used in the composition according to the present invention is chosen from a mixture of cetyl alcohol and cetearyl alcohol (cetearyl alcohol).

It is preferable that the (b) fatty alcohol be selected from the group consisting of cetyl alcohol, cetearyl alcohol, stearyl alcohol, behenyl alcohol, oleyl alcohol, and mixtures thereof.

The amount of the (b) fatty alcohol(s) in the composition according to the present invention may be 0.01% by weight or more, preferably 0.05% by weight or more, and more preferably 0.1% by weight or more, relative to the total weight of the composition.

On the other hand, the amount of the (b) fatty alcohol(s) in the composition according to the present invention may be 15% by weight or less, preferably 10% by weight or less, and more preferably 5% by weight or less, relative to the total weight of the composition.

The amount of the (b) fatty alcohol(s) in the composition according to the present invention may 50 range from 0.01% to 15% by weight, preferably from 0.05% to 10% by weight, and more preferably from 0.1% to 5% by weight, relative to the total weight of the composition.

### [Acid]

The composition according to the present invention comprises (c) at least one acid selected from hydroxy acids, phytic acid, and mixtures thereof. If two or more such acids are used, they may be the same or different.

The (c) acid can function as a peeling agent, in particular a peeling agent for the desquamation of a keratin layer such as the stratum corneum of the skin.

It is preferable that the hydroxyl acid be selected from alpha-hydroxy acids, beta-hydroxy acids, and mixtures thereof.

The term “alpha-hydroxy acid”, or “AHA”, here means a carboxylic acid which has at least one carboxyl group and at least one hydroxyl group separated by one carbon atom. Thus, in other words, AHA is a carboxylic acid which has at least one hydroxyl group on the adjacent (alpha) carbon atom.

The alpha-hydroxy acid may be selected from, for example glycolic acid, lactic acid, malic acid, citric acid, mandelic acid, and mixtures thereof, preferably from glycolic acid, lactic acid, and mixtures thereof, and more preferably from lactic acid.

The term “alpha-hydroxy acids” here includes not only AIA but also derivatives of AHA. The derivatives of AHA may be, for example, esters of AHA which can release AHA by hydrolysis.

The term “beta-hydroxy acid”, or “BHA”, here means a carboxylic acid which has at least one carboxyl group and at least one hydroxyl group separated by two carbon atoms.

The beta-hydroxy acid may be salicylic acid.

The term “beta-hydroxy acids” here includes not only BHA but also derivatives of BHA. The derivatives of BHA may be, for example, esters of BHA which can release BHA by hydrolysis.

The (c) acid may be selected from the compounds (salicylic acid derivatives) represented by the following formula (I):

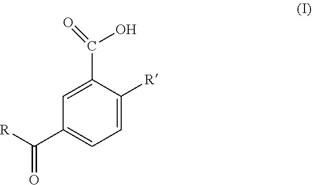

in which
   the radical R denotes a linear, branched or cyclic, saturated aliphatic chain containing from 2 to 22 carbon atoms; an unsaturated chain containing from 2 to 22 carbon atoms containing one or more double bonds that may be conjugated; an aromatic nucleus linked to the carbonyl radical directly or via saturated or unsaturated aliphatic chains containing from 2 to 7 carbon atoms; said groups possibly being substituted with one or more substituents, which may be identical or different, chosen from halogen atoms, a trifluoromethyl group, hydroxyl groups in free form or esterified with an acid containing from 1 to 6 carbon atoms, or carboxyl groups in free form or esterified with a lower alcohol containing from 1 to 6 carbon atoms;
   R′ is a hydroxyl group or an ester group of formula:

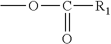

in which R₁ denotes a linear or branched, saturated or unsaturated aliphatic chain containing 1 to 18 carbon atoms; and
also salts thereof derived from an inorganic or organic base.

Preferably, the R radical in formula (I) denotes a linear, branched or cyclic, saturated aliphatic chain containing from 3 to 11 carbon atoms; an unsaturated chain containing from 3 to 17 carbon atoms and comprising one or more conjugated or unconjugated double bonds; it being possible for said hydrocarbon-based chains to be substituted with one or more substituents, which may be identical or different, chosen from halogen atoms, a trifluoromethyl group, hydroxyl groups in free form or esterified with an acid containing from 1 to 6 carbon atoms, or a carboxyl function in free form or esterified with a lower alcohol containing from 1 to 6 carbon atoms.

In one preferred embodiment, the R radical in formula (I) denotes a linear or branched alkyl or alkenyl group, preferably alkyl group, containing 2 or more, preferably 3 or more, more preferably 4 or more, even more preferably 5 or more, and in particular 6 or more carbon atoms, and/or 22 or less, preferably 18 or less, even more preferably 14 or less, preferentially 12 or less, and in particular 10 or less carbon atoms.

Preferentially, R′ in formula (I) is a hydroxyl group or an ester group of formula:

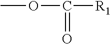

in which R₁ denotes a radical—(CH₂)ₙ—CH₃ where n is a number ranging from 0 to 14.

The compounds according to formula (I) in which the R radical is a C₃-C₁₀ alkyl group and/or R′ denotes hydroxyl may be preferable.

The compounds according to formula (I) in which R radical represents a chain derived from caprylic, linoleic, linolenic or oleic acid may also be preferable.

The compounds according to formula (I) in which the R radical denotes a C₃-C₁₀ alkyl group bearing a carboxyl function in free form or esterified with a lower alcohol containing from 1 to 6 carbon atoms and R′ denotes hydroxyl may also be preferable.

The compounds of formula (I) that may be used according to the present invention are in particular described in U.S. Pat. Nos. 6,159,479 and 5,558,871, FR 2,581,542, U.S. Pat. No. 4,767,750, EP 378 936, U.S. Pat. Nos. 5,267,407, 5,667,789, 5,580,549 and EP-A-570,230.

Among the particularly preferred compounds of formula (I), mention may be made of 5-n-octanoylsalicylic acid (or capryloyl salicylic acid); 5-n-decanoylsalicylic acid; 5-n-dodecanoylsalicylic acid; 5-n-heptyloxysalicylic acid, and the corresponding salts thereof. The derivative in question is preferably 5-n-octanoylsalicylic acid.

The (c) acid may be in the form of a salt. As the salts derived from inorganic bases, mention may particularly be made of those derived from alkali metal or alkaline-earth metal hydroxylated bases, for instance sodium hydroxide or potassium hydroxide, and ammonia. As regards the salts derived from the organic bases, mention may particularly be made of those derived from bases of amine or alkanolamine type.

It is preferable to use, as the (c) acids, a combination of at least one alpha-hydroxy acid and at least one beta-hydroxy acid, and more preferably a combination of lactic acid and salicylic acid, with phytic acid.

It is known that AHA activates the protease in the stratum corneum to enhance the degradation of corneodesmosomes, while in the dermis layer, the AHA can promote collagen formation, which subsequently leads to desquamation of skin. In addition, it has been reported that AHA also decreases facial pores and blackheads, most likely by targeting enzyme activation and protein functions linked to sebum production.

It is known that BHA adds keratolytic activity which promotes the peeling and shedding of cells in the stratum corneum layer. It also decongests the skin by softening the sebum which blocks the pores. For this reason, it is effective for improving desquamation and especially effective for acne and oily skin types as well.

The amount of the (c) acid(s) in the composition according to the present invention may be 0.01% by weight or more, preferably 0.1% by weight or more, and more preferably 1% by weight or more, relative to the total weight of the composition.

On the other hand, the amount of the (c) acid(s) in the composition according to the present invention may be 15% by weight or less, preferably 10% by weight or less, and more preferably 8% by weight or less, relative to the total weight of the composition.

The amount of the (c) acid(s) in the composition according to the present invention may be from 0.01% to 15% by weight, preferably from 0.1% to 10% by weight, and more preferably from 1% to 8% by weight, relative to the total weight of the composition.

### [Polyglyceryl Fatty Acid Ester]

The composition according to the present invention comprises (d) at least one polyglyceryl fatty acid ester having a C₆-C₃₂ fatty acid residue. A single type of such a polyglyceryl fatty acid ester may be used, but two or more different types of such polyglyceryl fatty acid esters may be used in combination.

The (d) polyglyceryl fatty acid ester having a C₆-C₃₂ fatty acid residue is a type of a nonionic surfactant.

It is preferable that the (d) polyglyceryl fatty acid ester having a C₆-C₃₂ fatty acid residue have a polyglycerol moiety derived from 2 to 10 glycerols, more preferably from 2 to 8 glycerols, and further more preferably from 2 to 6 glycerols.

If a single (d) polyglyceryl fatty acid ester having a C₆-C₃₂ fatty acid residue is used, the (d) polyglyceryl fatty acid ester having a C₆-C₃₂ fatty acid residue has an HLB (Hydrophilic Lipophilic Balance) value of from 8.0 to 11.5, preferably from 8.5 to 11.0, and more preferably from 9.0 to 10.5.

If two or more (d) polyglyceryl fatty acid esters having a C₆-C₃₂ fatty acid residue are used, the weighted average of HLB values of the (d) polyglyceryl fatty acid esters having a C₆-C₃₂ fatty acid residue is from 8.0 to 11.5, preferably from 8.5 to 11.0, and more preferably from 9.0 to 10.5.

The (d) polyglyceryl fatty acid ester having a C₆-C₃₂ fatty acid residue may be chosen from polyglyceryl fatty acid mono, di and tri esters. It is preferable that the (d) polyglyceryl fatty acid ester be selected from polyglyceryl fatty acid monoesters and diesters.

It is preferable that the ingredient (d) comprise at least one first polyglyceryl fatty acid ester having a C₆-C₂₂ fatty acid residue, and at least one second polyglyceryl fatty acid ester having a C₂₄-C₃₂ fatty acid residue.

The first polyglyceryl fatty acid ester has a C₆-C₂₂ fatty acid residue. The C₆-C₂₂ fatty acid residue may be derived from saturated or unsaturated fatty acids, preferably saturated fatty acids, including 6 to 22 carbon atoms, preferably 7 to 20 carbon atoms, and more preferably 8 to 18 carbon atoms, such as lauric acid, oleic acid, stearic acid, isostearic acid, capric acid, caprylic acid, and myristic acid.

The first polyglyceryl fatty acid ester having a C₆-C₂₂ fatty acid residue may be selected from the group consisting of PG2 caprylate, PG2 sesquicaprylate, PG2 dicaprylate, PG2 tricaprylate, PG2 caprate, PG2 sesquicaprate, PG2 dicaprate, PG2 tricaprate, PG2 laurate, PG2 sesquilaurate, PG2 dilaurate, PG2 trilaurate, PG2 myristate, PG2 sesquimyristate, PG2 dimyristate, PG2 trimyristate, PG2 stearate, PG2 sesquistearate, PG2 distearate, PG2 tristearate, PG2 isostearate, PG2 sesquiisostearate, PG2 diisostearate, PG2 triisostearate, PG2 oleate, PG2 sesquioleate, PG2 dioleate, PG2 trioleate, PG3 caprylate, PG3 sesquicaprylate, PG3 dicaprylate, PG3 tricaprylate, PG3 caprate, PG3 sesquicaprate, PG3 dicaprate, PG3 tricaprate, PG3 laurate, PG3 sesquilaurate, PG3 dilaurate, PG3 trilaurate, PG3 myristate, PG3 sesquimyristate, PG3 dimyristate, PG3 trimyristate, PG3 stearate, PG3 sesquistearate, PG3 distearate, PG3 tristearate, PG3 isostearate, PG3 sesquiisostearate, PG3 diisostearate, PG3 triisostearate, PG3 oleate, PG3 sesquioleate, PG3 dioleate, PG3 trioleate, PG4 caprylate, PG4 sesquicaprylate, PG4 dicaprylate, PG4 tricaprylate, PG4 caprate, PG4 sesquicaprate, PG4 dicaprate, PG4 tricaprate, PG4 laurate, PG4 sesquilaurate, PG4 dilaurate, PG4 trilaurate, PG4 myristate, PG4 sesquimyristate, PG4 dimyristate, PG4 trimyristate, PG4 stearate, PG4 sesquistearate, PG4 distearate, PG4 tristearate, PG4 isostearate, PG4 sesquiisostearate, PG4 diisostearate, PG4 triisostearate, PG4 oleate, PG4 sesquioleate, PG4 dioleate, PG4 trioleate, PG5 caprylate, PG5 sesquicaprylate, PG5 dicaprylate, PG5 tricaprylate, PG5 tetracaprylate, PG5 caprate, PG5 sesquicaprate, PG5 dicaprate, PG5 tricaprate, PG5 tetracaprate, PG5 laurate, PG5 sesquilaurate, PG5 dilaurate, PG5 trilaurate, PG5 tetralaurate, PG5 myristate, PG5 sesquimyristate, PG5 dimyristate, PG5 trimyristate, PG5 tetramyristate, PG5 stearate, PG5 sesquistearate, PG5 distearate, PG5 tristearate, PG5 tetrastearate, PG5 isostearate, PG5 sesquiisostearate, PG5 diisostearate, PG5 triisostearate, PG5 tetraisostearate, PG5 oleate, PG5 sesquioleate, PG5 dioleate, PG5 trioleate, PG5 tetraoleate, PG6 caprylate, PG6 sesquicaprylate, PG6 dicaprylate, PG6 tricaprylate, PG6 tetracaprylate, PG6 pentacaprylate, PG6 caprate, PG6 sesquicaprate, PG6 dicaprate, PG6 tricaprate, PG6 tetracaprate, PG6 pentacaprate, PG6 laurate, PG6 sesquilaurate, PG6 dilaurate, PG6 trilaurate, PG6 tetralaurate, PG6 pentalaurate, PG6 myristate, PG6 sesquimyristate, PG6 dimyristate, PG6 trimyristate, PG6 tetramyristate, PG6 pentamyristate, PG6 stearate, PG6 sesquistearate, PG6 distearate, PG6 tristearate, PG6 tetrastearate, PG6 pentastearate, PG6 isostearate, PG6 sesquiisostearate, PG6 diisostearate, PG6 triisostearate, PG6 tetraisostearate, PG6 pentaisostearate, PG6 oleate, PG6 sesquioleate, PG6 dioleate, PG6 trioleate, PG6 tetraoleate, PG6 pentaoleate, PG10 caprylate, PG10 sesquicaprylate, PG10 dicaprylate, PG10 tricaprylate, PG10 tetracaprylate, PG10 pentacaprylate, PG10 hexacaprylate, PG10 caprate, PG10 sesquicaprate, PG10 dicaprate, PG10 50 tricaprate, PG10 tetracaprate, PG10 pentacaprate, PG10 hexacaprate, PG10 laurate, PG10 sesquilaurate, PG10 dilaurate, PG10 trilaurate, PG10 tetralaurate, PG10 pentalaurate, PG10 hexalaurate, PG10 myristate, PG10 sesquimyristate, PG10 dimyristate, PG10 trimyristate, PG10 tetramyristate, PG10 pentamyristate, PG10 hexamyristate, PG10 stearate, PG10 sesquistearate, PG10 distearate, PG10 tristearate, PG10 tetrastearate, PG10 pentastearate, PG10 hexastearate, PG10 isostearate, PG10 sesquiisostearate, PG10 diisostearate, PG10 triisostearate, PG10 tetraisostearate, PG10 pentaisostearate, PG10 hexaisostearate, PG10 oleate, PG10 sesquioleate, PG10 dioleate, PG10 trioleate, PG10 tetraoleate, PG10 pentaoleate, and PG10 hexaoleate.

It is preferable that the first polyglyceryl fatty acid ester having a C₆-C₂₂ fatty acid residue be chosen from:
polyglyceryl monoisostearate comprising 2 to 6 glycerol units,
polyglyceryl monocaprate comprising 2 to 6 glycerol units,
polyglyceryl monooleate comprising 2 to 6 glycerol units, and
polyglyceryl distearate comprising 2 to 6 glycerol units.

As examples of the first polyglyceryl fatty acid ester having a C₆-C₂₂ fatty acid residue, mention may be made of Salacos 41V from Nisshin Oilio Group, Ltd., Tegosoft PC 41 from Evonik Co., Ltd., and Emalex DSG-6 from Nihon Emulsion Co., Ltd.

The second polyglyceryl fatty acid ester has a C₂₄-C₃₂ fatty acid residue. The C₂₄-C₃₂ fatty acid residue may be derived from saturated or unsaturated fatty acids, preferably saturated fatty acids, including 24 to 32 carbon atoms, preferably 25 to 30 carbon atoms, and more preferably 26 to 28 carbon atoms, such as lignoceric acid, nervonic acid, cerotic acid, and montanic acid.

It is preferable that the second polyglyceryl fatty acid ester having a C₂₄-C₃₂ fatty acid residue be a polyglyceryl diester represented by the following formula (B)

R³—C(O)—(O—CH₂—CH(OH)—CH₂)ₙ—O—C(O)—R⁴ (B)

wherein:
R³ and R⁴ denote, respectively, a linear or branched, saturated or unsaturated C₂₄ to C₃₂ fatty chain, and
n denotes 2 to 10, preferably 2 to 8 and more preferably 2 to 6.

According to an embodiment, in formula (B), the R³—C(O)— group corresponds to the carbon chain of a C₂₄ to C₃₂ fatty acid, said acid usually being linear and saturated, and preferably corresponds to a linear and saturated C₂₄ to C₃₀ fatty acid. This therefore includes, for example, tetracosanoic (or lignoceric) acid (C₂₄) hexacosanoic (or cerotic) acid (C₂₆). The R⁴ group corresponds to the hydrocarbon chain of an alcohol, said alcohol usually being saturated and linear, and having a C₂₄ to C₃₂ chain, preferably C₂₄ to C₃₀ chain.

According to the present invention, the polyglyceryl diester can be obtained by esterification of a solid wax in the presence of at least one polyol.

Depending on the source of the wax, the mixture of monoesters may also contain a certain proportion of hydroxy acid esters such as hydroxypalmitic or hydroxystearic acid. This is the case, for example, of beeswax. Preferably said alcohol is eicosanol, docosanol or tetracosanol. Beeswax, carnauba wax, candelilla wax, rice bran wax, sunflower wax, ouricury wax, Shellac wax and sugarcane wax are examples of natural solid waxes. Preferably, the solid wax is beeswax.

Solid waxes suitable for obtaining the polyglyceryl diester may have a melting point between 5° and 90° C. They correspond to mixtures essentially comprising monoesters having the formula R¹—C(O)—O—R², where the R¹—C(O)— group corresponds to the carbon chain of the fatty acid, said acid usually being linear and saturated and having a number of carbon atoms of at least 24, and in particular 26, and preferably up to 32 and more preferably 30. This therefore includes hexacosanoic (or cerotic) acid (C26). Depending on the source of the wax, the mixture of monoesters may also contain a certain proportion of hydroxy acid esters such as hydroxypalmitic or hydroxystearic acid. This is the case, for example, of beeswax. The R² group corresponds to the hydrocarbon chain of an alcohol, said alcohol usually being saturated and linear and having a number of carbon atoms of at least 18, and in particular 20, and preferably up to 44 and more preferably 34. Preferably said alcohol is eicosanol, docosanol or tetracosanol. Beeswax, carnauba wax, candelilla wax, rice bran wax, sunflower wax, ouricury wax, Shellac wax and sugarcane wax are examples of natural solid waxes.

Preferably, the solid wax suitable for the esterification reaction is beeswax.

Preferably, the polyol used for esterification is selected from the group comprising ethylene glycol, diethylene glycol, triethylene glycol, 2-methyl propanediol, propylene glycol, butylene glycol, neopentyl glycol, hexylene glycol, octylene glycol, polyethylene glycol, polypropylene glycol, trimethylol propane, sorbitol, erythritol, pentaerythritol, dipentaerythritol, glycerol, diglycerol and polyglycerol (i.e. a polymer of glycerol units). More preferably, the polyol is a polyglycerol, having an average degree of polymerization between 2 and 6, preferably of 3. Preferably, the polyol is polyglycerol-3.

The second polyglyceryl fatty acid ester having a C₂₄-C₃₂ fatty acid residue may comprise the acid part of a solid wax. Waxes have a complex composition. They have the common feature of containing a mixture of acid monoesters and very long chain fatty alcohols.

Preferably, the second polyglyceryl fatty acid ester having a C₂₄-C₃₂ fatty acid residue is a wax derivative obtained by reacting together at least one solid wax in the presence of at least one polyol and optionally at least one catalyst. In such a case, a transesterification reaction occurs between the various chemical entities yielding the wax derivative.

The preferred catalysts are hydroxides or alkaline or alkaline earth alkoxides, calcium hydroxide, potassium or sodium carbonates or catalysts based on tin or titanium.

Preferably, the solid wax is advantageously selected from the group comprising carnauba wax, candelilla wax, rice bran wax, sunflower wax, sugarcane wax, ouricury wax, beeswax and Shellac wax.

In a preferred embodiment, the wax derivative is obtained by reacting beeswax and a polyglycerol such as polyglycerol-3.

In practice, the reaction is preferably conducted at a temperature of between 100° C. and 220° C., advantageously between 150° C. and 200° C.

It is preferable that the second polyglyceryl fatty acid ester having a C₂₄-C₃₂ fatty acid residue be polyglyceryl-3 beeswax.

The second polyglyceryl fatty acid ester having a C₂₄-C₃₂ fatty acid residue may be used as a combination with other ingredients.

For example, the second polyglyceryl fatty acid ester having a C₂₄-C₃₂ fatty acid residue may be used as a combination of jojoba wax, cetyl alcohol, polyglyceryl-6 distearate, and polyglyceryl-3 beeswax.

As the second polyglyceryl fatty acid ester having a C₂₄-C₃₂ fatty acid residue, EMULIUM® MELLIFERA (Gattefossé) comprising a mixture of jojoba wax, cetyl alcohol, polyglyceryl-6 distearate, and polyglyceryl-3 beeswax (INCI name: Polyglyceryl-6 Distearate (and) Jojoba Esters (and) Polyglyceryl-3 Beeswax (and) Cetyl Alcohol) may be used. This mixture comprises from 5% to 30% by weight of jojoba wax; from 3% to 15% by weight of cetyl alcohol; at least 50% by weight of polyglyceryl-6 distearate; and from 3% to 15% by weight of polyglyceryl-3 beeswax, relative to the total weight of the mixture.

The amount of the (d) polyglyceryl fatty acid ester(s) having a C₆-C₃₂ fatty acid residue in the composition according to the present invention may be 0.01% by weight or more, preferably 0.05% by weight or more, and more preferably 0.1% by weight or more, relative to the total weight of the composition.

On the other hand, the amount of the (d) polyglyceryl fatty acid ester(s) having a C₆-C₃₂ fatty acid residue in the composition according to the present invention may be 20% by weight or less, preferably 15% by weight or less, and more preferably 10% by weight or less, relative to the total weight of the composition.

The amount of the (d) polyglyceryl fatty acid ester(s) having a C₆-C₃₂ fatty acid residue in the composition according to the present invention may range from 0.01% to 20% by weight, preferably from 0.05% to 15% by weight, more preferably from 0.1% to 10% by weight, relative to the total weight of the composition.

If the ingredient (d) comprises at least one first polyglyceryl fatty acid ester having a C₆-C₂₂ fatty acid residue, and at least one second polyglyceryl fatty acid ester having a C₂₄-C₃₂ fatty acid residue, the weight ratio of the amount of the first polyglyceryl fatty acid ester/the amount of the second polyglyceryl fatty acid ester may be more than 15 and less than 35, preferably more than 17 and less than 30, and more preferably more than 19 and less than 25.

### [Thickener]

The composition according to the present invention comprises (e) at least one thickener. A single type of thickener may be used, but two or more different types of thickeners may be used in combination.

The (e) thickener or thickening agent may be selected from organic and inorganic thickeners.

The (e) thickener may be selected from the group consisting of:
(i) associative thickeners;
(ii) crosslinked acrylic acid homopolymers;
(iii) crosslinked copolymers of (meth)acrylic acid and of (C₁-C₆)alkyl acrylate;
(iv) nonionic homopolymers and copolymers comprising ethylenically unsaturated monomers of ester and/or amide type;
(v) ammonium acrylate homopolymers and copolymers of ammonium acrylate and of acrylamide;
(vi) polysaccharides; and
(vii) C₁₂-C₃₀ fatty alcohols.
(i) As used herein, the expression “associative thickener” means an amphiphilic thickener comprising both hydrophilic units and hydrophobic units, for example, at least one C₈-C₃₀ fatty chain and at least one hydrophilic unit.

Representative associative thickeners that may be used are associative polymers chosen from:
(aa) nonionic amphiphilic polymers comprising at least one fatty chain and at least one hydrophilic unit;
(bb) anionic amphiphilic polymers comprising at least one hydrophilic unit and at least one fatty-chain unit;
(cc) cationic amphiphilic polymers comprising at least one hydrophilic unit and at least one fatty-chain unit; and
(dd) amphoteric amphiphilic polymers comprising at least one hydrophilic unit and at least one fatty-chain unit,
wherein the fatty chain contains from 10 to 30 carbon atoms.

The (aa) nonionic amphiphilic polymers comprising at least one fatty chain and at least one hydrophilic unit may, for example, be chosen from:
(1) celluloses modified with groups comprising at least one fatty chain; examples that may be mentioned include:
   hydroxyethylcelluloses modified with groups comprising at least one fatty chain chosen from alkyl, arylalkyl, and alkylaryl groups, and in which the alkyl groups are, for example, C₈-C₂₂, such as the product Natrosol Plus Grade 330 CS(C₁-C₆ alkyls) sold by the company Aqualon, and the product Bermocoll EHM 100 sold by the company Berol Nobel, and
   celluloses modified with polyalkylene glycol alkylphenyl ether groups, such as the product Amercell Polymer HM-1500 (polyethylene glycol (15) nonylphenyl ether) sold by the company Amerchol.
(2) hydroxypropyl guars modified with groups comprising at least one fatty chain, such as the product Esaflor HM 22 (C₂₂ alkyl chain) sold by the company Lamberti, and the products Miracare XC95-3 (C₁₄ alkyl chain) and RE205-1 (C₂₀ alkyl chain) sold by the company Rhodia Chimie.
(3) polyether-urethanes comprising at least one fatty chain, such as C₁₀-C₃₀ alkyl or alkenyl groups, for instance the products Elfacos T 210 and Elfacos T 212 sold by the company Akzo or the products Aculyn 44 and Aculyn 46 sold by the company Rohm & Haas.
(4) copolymers of vinylpyrrolidone and of hydrophobic fatty-chain monomers;
   examples that may be mentioned include:
   the products Antaron V216 and Ganex V216 (vinylpyrrolidone/hexadecene copolymer) sold by the company I.S.P., and
   the products Antaron V220 and Ganex V220 (vinylpyrrolidone/eicosene copolymer) sold by the company I.S.P.
(5) copolymers of C₁-C₆ alkyl acrylates or methacrylates and of amphiphilic monomers comprising at least one fatty chain, such as the oxyethylenated methyl
   methacrylate/stearyl acrylate copolymer sold by the company Goldschmidt under the name Antil 208.
(6) copolymers of hydrophilic acrylates or methacrylates and of hydrophobic monomers comprising at least one fatty chain, such as a polyethylene glycol methacrylate/lauryl methacrylate copolymer.

The (bb) anionic amphiphilic polymers comprising at least one hydrophilic unit and at least one fatty-chain unit, may, for example, be chosen from those comprising at least one fatty-chain allyl ether unit and at least one hydrophilic unit comprising an ethylenic unsaturated anionic monomeric unit, for example, a vinylcarboxylic acid unit and further, for example, be chosen from units derived from acrylic acids, methacrylic acids, and mixtures thereof, wherein the fatty-chain allyl ether unit corresponds to the monomer of formula (I) below:

CH₂═C(R₁)CH₂OBₙR (I)

in which R₁ is chosen from H and CH₃, B is an ethyleneoxy radical, n is chosen from zero and integers ranging from 1 to 100, R is chosen from hydrocarbon-based radicals chosen from alkyl, arylalkyl, aryl, alkylaryl, and cycloalkyl radicals, containing from 10 to 30 carbon atoms, and further, for example, from 10 to 24 carbon atoms and even further, for example, from 12 to 18 carbon atoms.

In one embodiment, a unit of formula (I) is, for example, a unit in which R₁ can be H, n can be equal to 10, and R can be a stearyl (C₁₈) radical.

Anionic amphiphilic polymers of this type are described and prepared, according to an emulsion polymerization process, in patent EP-0 216 479 B2.

In one embodiment, anionic amphiphilic polymers are, for example, polymers formed from 20% to 60% by weight of acrylic acid and/or of methacrylic acid, from 5% to 60% by weight of lower alkyl (meth)acrylates, from 2% to 50% by weight of fatty-chain allyl ether of formula (I), and from 0% to 1% by weight of a crosslinking agent which is a well-known copolymerizable unsaturated polyethylenic monomer, for example, diallyl phthalate, allyl(meth)acrylate, divinylbenzene, (poly)ethylene glycol dimethacrylate, and methylenebisacrylamide.

Examples of such polymers are crosslinked terpolymers of methacrylic acid, of ethyl acrylate, and of polyethylene glycol (10 EO) stearyl ether (Steareth-10), such as those sold by the company Ciba under the names Salcare SC 80 and Salcare SC 90, which are aqueous 30% emulsions of a crosslinked terpolymer of methacrylic acid, of ethyl acrylate, and of steareth-10 allyl ether (40/50/10).

The anionic amphiphilic polymers may further be chosen, for example, from those comprising at least one hydrophilic unit of unsaturated olefinic carboxylic acid type, and at least one hydrophobic unit of a type such as a (C₁₀-C₃₀) alkyl ester of an unsaturated carboxylic acid. The hydrophilic unit of unsaturated olefinic carboxylic acid type corresponds to, for example, the monomer of formula (II) below:

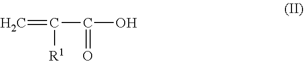

in which R¹ is chosen from H, CH₃, and C₂H₅, i.e., acrylic acid, methacrylic acid, and methacrylic acid units. The hydrophobic unit of a type such as a (C₁₀-C₃₀) alkyl ester of an unsaturated carboxylic acid corresponds to, for example, the monomer of formula (III) below:

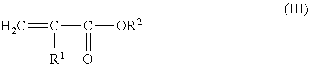

in which R¹ is chosen from H, CH₃, and C₂H₅(i.e., acrylate, methacrylate, and methacrylate units) and is, for example, chosen from, for example, H (acrylate units) and CH₃ (methacrylate units), and R² is chosen from C₁₀-C₃₀ alkyl radicals, for example, C₁₂-C₂₂ alkyl radicals.

Examples of (C₁₀-C₃₀)alkyl esters of unsaturated carboxylic acids include lauryl acrylate, stearyl acrylate, decyl acrylate, isodecyl acrylate, and dodecyl acrylate, and the corresponding methacrylates, lauryl methacrylate, stearyl methacrylate, decyl methacrylate, isodecyl methacrylate, and dodecyl methacrylate.

Anionic amphiphilic polymers of this type are disclosed and prepared, for example, according to U.S. Pat. Nos. 3,915,921 and 4,509,949.

Representative anionic amphiphilic polymers that can be used may further be chosen from polymers formed from a mixture of monomers comprising:
(7) acrylic acid, an ester of formula (IV) below:

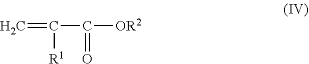

in which R¹ is chosen from H and CH₃, R² is chosen from C₁₀-C₃₀ alkyl radicals, such as alkyl radicals containing from 12 to 22 carbon atoms, and a crosslinking agent; such as polymers derived from 95% to 60% by weight of the acrylic acid (hydrophilic unit), 4% to 40% by weight of C₁₀-C₃₀ alkyl acrylate (hydrophobic unit), and 0% to 6% by weight of crosslinking polymerizable monomer, or polymers derived from 98% to 96% by weight of the acrylic acid (hydrophilic unit), 1% to 4% by weight of C₁₀-C₃₀ alkyl acrylate (hydrophobic unit), and 0.1% to 0.6% by weight of crosslinking polymerizable monomer; or
(8) acrylic acid and lauryl methacrylate, such as the polymers formed from 66% by weight of acrylic acid and 34% by weight of lauryl methacrylate.

The crosslinking agent can be a monomer comprising the group

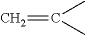

with at least one other polymerizable group whose unsaturated bonds are not conjugated.

Mention may be made, for example, of polyallyl ethers such as polyallylsucrose and polyallylpentaerythritol.

Among said polymers above, mention may be made, for example, of the products sold by the company Goodrich under the trade names Pemulen TR1, Pemulen TR2, and Carbopol 1382, and further, for example, Pemulen TR1, and the product sold by the company S.E.P.C. under the name Coatex SX.

Among anionic amphiphilic fatty-chain polymers, mention may also be made, for example, of the ethoxylated copolymer of methacrylic acid/methyl acrylate/alkyl dimethyl-meta-isopropenylbenzylisocyanate sold under the name Viscophobe DB 1000 by the company Amerchol.

The (cc) cationic amphiphilic polymers used are, for example, chosen from quaternized cellulose derivatives and polyacrylates comprising amino side groups.

The quaternized cellulose derivatives are, for example, chosen from quaternized celluloses modified with groups comprising at least one fatty chain, such as alkyl, arylalkyl, and alkylaryl groups comprising at least 8 carbon atoms, and mixtures thereof, and quaternized hydroxyethylcelluloses modified with groups comprising at least one fatty chain, such as alkyl, arylalkyl, and alkylaryl groups comprising at least 8 carbon atoms, and mixtures thereof.

Quaternized and non-quaternized polyacrylates comprising amino side groups have, for example, hydrophobic groups, such as Steareth 20 (polyoxy-ethylenated (20) stearyl alcohol) and (C₁₀-C₃₀)alkyl PEG-20 itaconate.

The alkyl radicals borne by the above quaternized celluloses and hydroxyethylcelluloses, for example, contain from 8 to 30 carbon atoms.

The aryl radicals, for example, are chosen from phenyl, benzyl, naphthyl, and anthryl groups.

Examples of quaternized alkylhydroxyethyl-celluloses comprising C₈-C₃₀ fatty chains are the products Quatrisoft LM 200, Quatrisoft LM-X 529-18-A, Quatrisoft LM-X 529-18B (C₁₂ alkyl), and Quatrisoft LM-X 529-8 (C₁₈ alkyl) sold by the company Amerchol, and the products Crodacel QM, Crodacel QL (C₁₂ alkyl), and Crodacel QS (C₁₈ alkyl) sold by the company Croda.

Examples of polyacrylates comprising amino side chains are the polymers 8781-124B or 9492-103 and Structure Plus from the company National Starch.

Among the (dd) amphoteric amphiphilic polymers comprising at least one hydrophilic unit and at least one fatty-chain unit, mention may be made, for example, of copolymers of methacrylamidopropyltrimethylammonium chloride/acrylic acid/C₁₀-C₃₀ alkyl methacrylate, wherein the alkyl radical is, for example, a stearyl radical.

The associative thickeners in the compositions can have, for example, in solution or in dispersion at a concentration of 1% active material in water, a viscosity, measured using a Rheomat RM 180 rheometer at 25° C., of greater than 0.1 cps and further, for example, of greater than 0.2 cps, at a shear rate of 200 s⁻¹.

The associative thickener may be an associative polymeric thickener, preferably an associative polyurethane thickener.

The associative polyurethane thickener may be cationic or nonionic.

Among the associative polyurethane thickeners, there may be mention of the associative polyurethane derivatives such as those obtained by polymerization: about 20% to 70% by weight of a carboxylic acid containing an α,β-monoethylenic unsaturation, about 20 to 80% by weight of a nonsurfactant monomer containing an α,β-monoethylenic unsaturation, about 0.5 to 60% by weight of a nonionic mono-urethane which is the product of the reaction of a monohydroxylated surfactant with a monoethylenically unsaturated monoisocyanate.

The like are described in particular in EP 173109 and more particularly in Example 3 thereof. More precisely, this polymer is a methacrylic acid/methyl acrylate/dimethyl metaisopropenyl benzyl isocyanate of ethoxylated behenyl alcohol (40 EO) terpolymer as an aqueous dispersion at 25%. This product is provided under the reference VISCOPHOBE DB1000 by the company AMERCHOL.

Also suitable are the cationic associative polyurethane thickeners the family of which has been described by the applicant in French Patent Application No. 0009609. They can be represented more particularly by the following general formula (A): R—X—(P)ₙ-[L-(Y)ₘ]ᵣ-L′-(P′)ₚ—X′—R′(A) in which: R and R′, which are identical or different, represent a hydrophobic group or a hydrogen atom; X and X′, which are identical or different, represent a group containing an amine functional group carrying or otherwise a hydrophobic group, or alternatively the group L″; L, L′ and L″, which are identical or different, represent a group derived from a diisocyanate; P and P′, which are identical or different, represent a group containing an amine functional group carrying or otherwise a hydrophobic group; Y represents a hydrophilic group; r is an integer between 1 and 100, preferably between 1 and 50 and in particular between 1 and 25; n, m and p are each independently of the others between 0 and 1000; the molecule containing at least one protonated or quaternized amine functional group and at least one hydrophobic group.

In a very advantageous embodiment, the only hydrophobic groups of these polyurethanes are the groups R and R′ at the chain ends.

According to a first preferred embodiment, the associative polyurethane thickener corresponds to formula (A) in which R and R′ both represent independently a hydrophobic group, X, X′ each represent a group L″, n and p are between 1 and 1000, and L, L′, L″, P, P′, Y and in have the meaning indicated in formula (A).

According to another preferred embodiment of the present invention, the associative polyurethane thickener corresponds to formula (A) in which R and R′ both represent independently a hydrophobic group, X, X′ each represent a group L″, n and p are equal to 0, and L, L′, L″, Y and m have the meaning in formula (A) indicated above.

The fact that n and p are equal to 0 means that these polymers do not contain units derived from a monomer containing an amine functional group, incorporated into the polymer during polycondensation. The protonated amine functional groups of these polyurethanes result from the hydrolysis of isocyanate functional groups, in excess, at the chain end, followed by alkylation of the primary amine functional groups formed by alkylating agents containing a hydrophobic group, that is to say compounds of the RQ or R′Q type, in which R and R′ are as defined above and Q denotes a leaving group such as a halide, a sulfate and the like.

In accordance with another preferred embodiment of the present invention, the associative polyurethane thickener corresponds to formula (A) in which R and R′ both represent independently a hydrophobic group, X and X both represent independently a group containing a quaternary amine, n and p are equal to zero, and L, L′, Y and m have the meaning indicated in formula (A).

The number-average molecular mass of the cationic associative polyurethane thickeners is usually between 400 and 500,000, in particular between 1,000 and 400,000, and ideally between 1,000 and 300,000 g/mol.

When X and/or X′ denote a group containing a tertiary or quaternary amine, X and/or X′ may represent one of the following formulae:

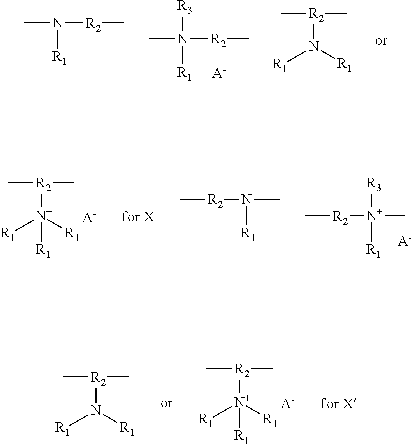

in which:
R₂ represents a linear or branched alkylene radical having from 1 to 20 carbon atoms, containing or otherwise a saturated or unsaturated ring, or an arylene radical, it being possible for one or more of the carbon atoms to be replaced by a heteroatom chosen from N, S, O, P;
R₁ and R₃, which are identical or different, denote a linear or branched, C₁-C₃₀ alkyl or alkenyl radical, an aryl radical, it being possible for at least one of the carbon atoms to be replaced by a heteroatom chosen from N, S, O, and P;
A⁻ is a physiologically acceptable counterion.

The groups L, L′ and L″ represent a group of formula:

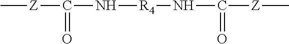

in which:
Z represents —O—, —S— or —NH—; and
R₄ represents a linear or branched alkylene radical having from 1 to 20 carbon atoms, containing or otherwise a saturated or unsaturated ring, an arylene radical, it being possible for one or more of the carbon atoms to be replaced by a heteroatom chosen from N, S, O and P.

The groups P and P′, comprising an amine functional group, may represent at least one of the following formulae:

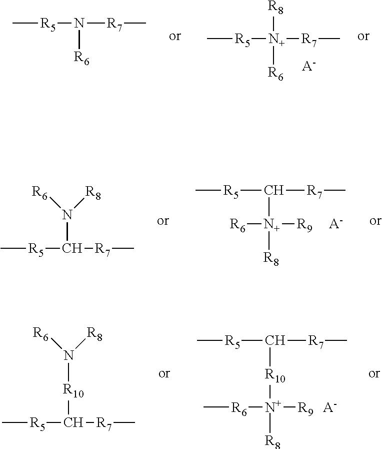

in which:
R₅ and R₇ have the same meanings as R₂ defined above; R₆, R₈ and R₉ have the same meanings as
R₁ and R₃ defined above;
R₁₀ represents a linear or branched alkylene group, which is optionally unsaturated and which may contain one or more heteroatoms chosen from N, O, S and P;
A⁻ is a physiologically acceptable counterion.

As regards the meaning of Y, the expression hydrophilic group is understood to mean a polymeric or nonpolymeric water-soluble group. By way of example, there may be mentioned, when polymers are not involved, ethylene glycol, diethylene glycol and propylene glycol. In accordance with a preferred embodiment, in the case of a hydrophilic polymer, there may be mentioned, by way of example, polyethers, sulfonated polyesters, sulfonated polyamides, or a mixture of these polymers. Preferably, the hydrophilic compound is a polyether and in particular a polyethylene oxide or a polypropylene oxide.

The cationic associative polyurethane thickeners of formula (A) are formed from diisocyanates and from various compounds possessing functional groups containing a labile hydrogen. The functional groups containing a labile hydrogen may be alcohol functional groups, primary or secondary amine functional groups or thiol functional groups which give, after reaction with the diisocyanate functional groups, polyurethanes, polyureas and polythioureas, respectively. The term “polyurethanes” of the present invention covers these three types of polymers, namely polyurethanes proper, polyureas and polythioureas and copolymers thereof.

A first type of compounds entering into the preparation of the polyurethane of formula (A) is a compound containing at least one unit containing an amine functional group. This compound may be multifunctional, but preferably the compound is difunctional, that is to say, according to a preferred embodiment, this compound contains two labile hydrogen atoms carried for example by a hydroxyl, primary amine, secondary amine or thiol functional group. It is also possible to use a mixture of multifunctional and difunctional compounds in which the percentage of multifunctional compounds is low.

As indicated above, this compound may contain more than one unit containing an amine functional group. It is then a polymer carrying a repeat of the unit containing an amine functional group.

This type of compound may be represented by one of the following formulae: HZ—(P)ₙ—ZH, or HZ—(P′)ₚ—ZH, in which Z, P, P′, n and p are as defined above.

By way of example of a compound containing an amine functional group, there may be mentioned N-methyldiethanolamine, N-tert-butyldiethanolamine, and N-sulfoethyldiethanolamine.

The second compound entering into the preparation of the polyurethane of formula (A) is a diisocyanate corresponding to the formula O═C═N—R₄—N═C═O in which R₄ is defined above.

By way of example, there may be mentioned methylenediphenyl diisocyanate, methylenecyclohexane diisocyanate, isophorone diisocyanate, toluene diisocyanate, naphthalene diisocyanate, butane diisocyanate, and hexane diisocyanate.

A third compound entering into the preparation of the polyurethane of formula (A) is a hydrophobic compound intended to form the terminal hydrophobic groups of the polymer of formula (A).

This compound consists of a hydrophobic group and a functional group containing a labile hydrogen, for example a hydroxyl, primary or secondary amine, or thiol functional group.

By way of example, this compound may be a fatty alcohol, such as, in particular, stearyl alcohol, dodecyl alcohol, and decyl alcohol. When this compound contains a polymeric chain, it may be for example a hydroxyl hydrogenated polybutadiene.

The hydrophobic group of the polyurethane of formula (A) may also result from the quaternization reaction of the tertiary amine of the compound containing at least one tertiary amine unit. Thus, the hydrophobic group is introduced by the quaternizing agent. This quaternizing agent is a compound of the RQ or R′Q type, in which R and R′ are as defined above and Q denotes a leaving group such as a halide, a sulfate, and the like.

The cationic associative polyurethane thickener may additionally comprise a hydrophilic sequence. This sequence is provided by a fourth type of compound entering into the preparation of the polymer. This compound may be multifunctional. It is preferably difunctional. It is also possible to have a mixture where the percentage of multifunctional compound is low.

The functional groups containing a labile hydrogen are alcohol, primary or secondary amine, or thiol functional groups. This compound may be a polymer terminated at the chain ends by one of these functional groups containing a labile hydrogen.

By way of example, there may be mentioned, when polymers are not involved, ethylene glycol, diethylene glycol and propylene glycol.

In the case of a hydrophilic polymer, there may be mentioned, by way of example, polyethers, sulfonated polyesters, sulfonated polyamides, or a mixture of these polymers. Preferably, the hydrophilic compound is a polyether and in particular a polyethylene oxide or a polypropylene oxide.

The hydrophilic group noted Y in formula (A) is optional. Indeed, the units containing a quaternary or protonated amine functional group may suffice to provide the solubility or water-dispersibility necessary for this type of polymer in an aqueous solution. Although the presence of a hydrophilic group Y is optional, cationic associative polyurethane thickeners which contain such a group are nevertheless preferred.

The associative polyurethane thickener used in the present invention may also be nonionic, in particular nonionic polyurethane-polyethers. The nonionic polyurethane-polyethers may have both at least one hydrophilic moiety and at least one hydrophobic moiety. More particularly, said polymers may contain in their chain both hydrophilic sequences most often of a polyoxyethylenated nature and hydrophobic sequences which may be aliphatic linkages alone and/or cycloaliphatic and/or aromatic linkages.

Preferably, these polyether-polyurethanes comprise at least two lipophilic hydrocarbon chains, having from 6 to 30 carbon atoms, preferably from 6 to 20, separated by a hydrophilic sequence, it being possible for the hydrocarbon chains to be pendent chains or chains at the end of a hydrophilic sequence. In particular, it is possible for one or more pendent chains to be envisaged. In addition, the polymer may comprise a hydrocarbon chain at one end or at both ends of a hydrophilic sequence.

The polyether-polyurethanes may be polyblocks, in particular in triblock form. The hydrophobic sequences may be at each end of the chain (for example: triblock copolymer with hydrophilic central sequence) or distributed both at the ends and in the chain (polyblock copolymers for example). These same polymers may also be in the form of graft units or may be star-shaped.

The associative polyurethane thickener can form a network in water in which the hydrophobic part connects quasi-micelles.

Therefore, the associative polyurethane thickeners can increase the viscosity or consistency of the composition according to the present invention. Thus, after application of the composition according to the present invention, the original elasticity of the composition can be recovered quickly.

The nonionic polyether-polyurethanes containing a fatty chain may be triblock copolymers whose hydrophilic sequence is a polyoxyethylenated chain comprising from 50 to 1000 oxyethylenated groups.

The nonionic polyether-polyurethanes comprise a urethane bond between the hydrophilic sequences, hence the origin of the name.

By extension, those whose hydrophilic sequences are linked by other chemical bonds to the hydrophobic sequences are also included among the nonionic polyether-polyurethanes containing a hydrophobic chain.

By way of example of nonionic polyether-polyurethanes containing a hydrophobic chain which can be used in the present invention, it is also possible to use Rheolate® 205 containing a urea functional group sold by the company RHEOX or else the Rheolates® 208, 204 or 212, as well as Acrysol RM 184®.

There may also be mentioned the product ELFACOS T210® containing a C₁₂-C₁₄ alkyl chain and the product ELFACOS T212® containing a C₁₈ alkyl chain from AKZO.

The product DW 1206B® from ROHM & HAAS containing a C₂₀ alkyl chain and with a urethane bond, sold at 20% dry matter content in water, may also be used.

It is also possible to use solutions or dispersions of these polymers in particular in water or in an aqueous-alcoholic medium. By way of example of such polymers, there may be mentioned Rheolate® 255, Rheolate® 278 and Rheolate® 244 sold by the company RHEOX. It is also possible to use the product DW 1206F and DW 1206J provided by the company ROHM & HAAS.

The above-described polyether-polyurethanes which can be used can also be chosen from those described in the article by G. Fonnum, J. Bakke and Fk. Hansen-Colloid Polym. Sci 271, 380-389 (1993).

As the above-described polyether-polyurethanes, mention may be made of polyurethane-polyethers comprising in their chain at least one polyoxyethylenated hydrophilic block and at least one of hydrophobic blocks containing at least one sequence chosen from aliphatic sequences, cycloaliphatic sequences, and aromatic sequences.

It is preferable that the polyurethane-polyethers comprise at least two hydrocarbon-based lipophilic chains having from 8 to 30 carbon atoms, separated by a hydrophilic block, and wherein the hydrocarbon-based chains are chosen from pendent chains and chains at the end of the hydrophilic block.

According to a specific form of the present invention, use will be made of a polyurethane/polyether that may be obtained by polycondensation of at least three compounds comprising (i) at least one polyethylene glycol comprising from 150 to 180 mol of ethylene oxide, (ii) a polyoxyethylenated stearyl alcohol comprising 100 mol of ethylene oxide, and (iii) a diisocyanate.

Such polyurethane/polyethers are sold especially by the company Elementis under the name Rheolate FX 1100® and Rheoluxe 811®, which is a polycondensate of polyethylene glycol containing 136 mol of ethylene oxide, of stearyl alcohol polyoxyethylenated with 100 mol of ethylene oxide and of hexamethylene diisocyanate (HDI) with a weight-average molecular weight of 40,000 (INCI name: PEG-136/Steareth-100/HDI Copolymer).

According to another specific form of the present invention, use will be made of a polyurethane/polyether that may be obtained by polycondensation of at least three compounds comprising (i) at least one polyethylene glycol comprising from 150 to 180 mol of ethylene oxide, (ii) stearyl alcohol or decyl alcohol, and (iii) at least one diisocyanate.

Such polyurethane/polyethers are sold in particular by the company Rohm & Haas under the names Aculyn 46® and Aculyn 44®.

Aculyn 46® having the INCI name: PEG-150/Stearyl Alcohol/SMDI Copolymer, is a polycondensate of polyethylene glycol comprising 150 or 180 mol of ethylene oxide, of stearyl alcohol and of methylenebis(4-cyclohexyl isocyanate) (SMDI) at 15% by weight in a matrix of maltodextrin (4%) and water (81%) (INCI name: PEG-150/Stearyl Alcohol/SMDI Copolymer).

Aculyn 44® (PEG-150/Decyl Alcohol/SMDI Copolymer) is a polycondensate of polyethylene glycol comprising 150 or 180 mol of ethylene oxide, of decyl alcohol and of methylenebis(4-cyclohexyl isocyanate) (SMDI) at 35% by weight in a mixture of propylene glycol (39%) and water (26%) (INCI name: PEG-150/Decyl Alcohol/SMDI Copolymer).

As the associative polyurethanes, it is preferable to use a compound represented by the following formula (1):

R¹—{(O—R²)ₖ—OCONH—R³[—NHCOO—(R⁴—O)ₙ—R⁵]ₕ}ₘ (1)

wherein R¹ represents a hydrocarbon group, R² and R⁴ independently represent alkylene groups having 2 to 4 carbon atoms, which alkylene groups may be identical or different from each other, or a phenylethylene group, R³ represents a hydrocarbon group, which may optionally have a urethane bond, R⁵ represents a branched chain or secondary hydrocarbon group, m represents a number of at least 2, h represents a number of at least 1, k represents a number within the range of 1 to 500, and n represents a number within the range of 1 to 200.

The hydrophobically modified polyurethane that is represented by the general formula (1) shown above is obtained by, for example, reacting at least one polyether polyol that is represented by the formula R¹—[(O—R²)ₖ—OH]ₘ, at least one polyisocyanate that is represented by the formula R³—(NCO)ₕ₊₁, and at least one monoalcohol that is represented by the formula HO—(R⁴—O)ₙ—R⁵.

In such cases, R¹ to R⁵ in the general formula (1) are determined by the compounds R¹—[(O—R²)ₖ—OH]ₘ, R³—(NCO)ₕ₊₁ and HO—(R⁴—O)ₙ—R⁵. The loading ratios among the three compounds are not particularly limited and should preferably be such that the ratio of the isocyanate group derived from the polyisocyanate to the hydroxyl group derived from the polyether polyol and the polyether monoalcohol is selected within the range of NCO/OH of between 0.8:1 and 1.4:1.

The polyether polyol compound that is represented by the formula R¹—[(O—R²)ₖ—OH]ₘ and that may be used preferably for obtaining the associative thickener represented by the general formula (1) may be obtained from addition polymerization of an m-hydric polyol with an alkylene oxide, such as ethylene oxide, propylene oxide, butylene oxide, or epichlorohydrin, or with styrene oxide, and the like.

The polyols should preferably be di- to octa-hydric polyols. Examples of the di- to octa-hydric polyols include dihydric alcohols, such as ethylene glycol, propylene glycol, butylene glycol, hexamethylene glycol, and neopentyl glycol; trihydric alcohols, such as glycerol, trioxy isobutane, 1,2,3-butanetriol, 1,2,3-pentanetriol, 2-methyl-1,2,3-propanetriol, 2-methyl-2,3,4-butanetriol, 2-ethyl-1,2,3-butanetriol, 2,3,4-pentanetriol, 2,3,4-hexanetriol, 4-propyl-3,4,5-heptanetriol, 2,4-dimethyl-2,3,4-pentanetriol, pentamethylglycerol, pentaglycerol, 1,2,4-butanetriol, 1,2,4-pentanetriol, trimethylolethane, and trimethylolpropane; tetrahydric alcohols, such as pentaerythritol, 1,2,3,4-pentanetetrol, 2,3,4,5-hexanetetrol, 1,2,4,5-pentanetetrol, and 1,3,4,5-hexanetetrol; pentahydric alcohols, such as adonitol, arabitol, and xylitol; hexahydric alcohols, such as dipentaerythritol, sorbitol, mannitol, and iditol; and octahydric alcohols, such as sucrose.

Also, R² is determined by the alkylene oxide, styrene oxide, or the like, which is subjected to the addition. Particularly, for availability and excellent effects, an alkylene oxide having 2 to 4 carbon atoms, or styrene oxide is preferable.

The alkylene oxide, styrene oxide, or the like, to be subjected to the addition may be subjected to single polymerization, or random polymerization or block polymerization of at least two members. The procedure for the addition may be a conventional procedure. Also, the polymerization degree k may be selected within the range of 0 to 1,000, preferably within the range of 1 to 500, and more preferably within the range of 10 to 200. Further, the ratio of the 50 ethylene group occupying R² should preferably be within the range of 50 to 100 mass % with respect to the total quantity of R². In such cases, the associative thickener appropriate for the purposes of the present invention is obtained.

Furthermore, the molecular weight of the polyether polyol compound that is represented by the formula R¹[(O—R²)ₖ—OH]ₘ should preferably be selected within the range of 500 to 100,000, and should more preferably be selected within the range of 1,000 to 50,000.

The polyisocyanate that is represented by the formula R³—(NCO)ₕ₊₁ and that may be used preferably for obtaining the hydrophobically modified polyether urethane represented by the general formula (1) employed in accordance with the present invention is not limited particularly in so far as the polyisocyanate has at least two isocyanate groups in the molecule. Examples of the polyisocyanates include aliphatic diisocyanates, aromatic diisocyanates, alicyclic diisocyanates, biphenyl diisocyanate, phenylmethane diisocyanate, phenylmethane triisocyanate, and phenylmethane tetraisocyanate.

Also, it is possible to employ dimers and trimers (isocyanurate bonds) of the above-enumerated polyisocyanates. Further, it is possible to employ a biuret obtained by a reaction with an amine.

Furthermore, it is possible to employ a polyisocyanate having a urethane bond obtained by a reaction of the aforesaid polyisocyanate compound and a polyol. As the polyol, di- to octa-hydric polyols are preferable, and the above-enumerated polyols are preferable. In cases where a tri- or higher-hydric polyisocyanate is used as the polyisocyanate that is represented by the formula R³—(NCO)ₙ₊ᵢ, it is preferable to employ the aforesaid polyisocyanate having the urethane bond.

The polyether monoalcohol that is represented by the formula HO—(R⁴—O)ₙ—R⁵ and that may be used preferably for obtaining the hydrophobically modified polyether urethane represented by the general formula (1) employed in accordance with the present invention is not limited particularly in so far as the polyether monoalcohol is a polyether of a straight chain, branched chain, or secondary monohydric alcohol. The polyether monoalcohol may be obtained by addition polymerization of the straight chain, branched chain, or secondary monohydric alcohol with an alkylene oxide, such as ethylene oxide, propylene oxide, butylene oxide, or epichlorohydrin, or with styrene oxide, and the like.

The compound represented by the general formula (1) may be produced by, for example, heating at a temperature of 80 to 90° C. for 1 to 3 hours and thereby causing a reaction to occur in the same manner as that in the ordinary reaction of a polyether and an isocyanate.

As the compound represented by the general formula (1), polyethyleneglycol-240/decyltetradeceth-20/hexamethylene diisocyanate copolymer is preferable. The polyethyleneglycol-240/decyltetradeceth-20/hexamethylene diisocyanate copolymer is also referred to as PEG-240/HDI copolymer bis-decyltetradeceth-20 ether.

According to the present invention, it is preferable that the associative polyurethane thickener be selected from Steareth-100/PEG-136/HDI Copolymer sold by the company Rheox under the name of Rheolate FX 1100, PEG-240/HDI Copolymer Bis-decyltetradeceth-20 ether sold by the company Asahi Denka under the name of Adekanol GT-700, and mixtures thereof.

(ii) Among the crosslinked acrylic acid homopolymers that may be mentioned are those 50 crosslinked with an allylic alcohol ether of the sugar series. Mention may be made of carbomer, which is a homopolymer of acrylic acid crosslinked with an allyl ether of pentaerythritol, an allyl ether of sucrose, or an allyl ether of propylene, such as the products sold under the names Carbopol 980, 981, 954, 2984, and 5984 by the company Lubrizol or the products sold under the names Synthalen M and Synthalen K by the company 3 VSA.

(iii) The crosslinked copolymers of (meth)acrylic acid and of C₁-C₆ alkyl acrylate can be chosen from crosslinked copolymers of methacrylic acid and of ethyl acrylate as an aqueous dispersion comprising 38% active material sold, for example, under the name Viscoatex 538C by the company Coatex, and crosslinked copolymers of acrylic acid and of ethyl acrylate as an aqueous dispersion comprising 28% active material sold under the name Aculyn 33 by the company Rohm & Haas. Crosslinked copolymers of methacrylic acid and of ethyl acrylate include an aqueous dispersion comprising 30% active material sold under the name CARBOPOL AQUA SF-1 by the company NOVEON.

(iv) Among the nonionic homopolymers or copolymers comprising ethylenically unsaturated monomers of ester and/or amide type, mention may be made of the products sold under the names: Cyanamer P250 by the company Cytec (polyacrylamide); PMMA MBX-8C by the company US Cosmetics (methyl methacrylate/ethylene glycol dimethacrylate copolymer); Acryloid B66 by the company Rohm & Haas (butyl methacrylate/methyl methacrylate copolymer); and BPA 500 by the company Kobo (polymethyl methacrylate).

(v) Ammonium acrylate homopolymers that may be mentioned include the product sold under the name Microsap PAS 5193 by the company Hoechst.

Copolymers of ammonium acrylate and of acrylamide include the product sold under the name Bozepol C Nouveau or the product PAS 5193 sold by the company Hoechst (which are described and prepared in documents FR-2 416 723, U.S. Pat. Nos. 2,798,053, and 2,923,692).

(vi) The polysaccharides are, for example, chosen from glucans, modified and unmodified starches (such as those derived, for example, from cereals, for instance wheat, corn, or rice, from vegetables, for instance yellow peas, and tubers, for instance potatoes or cassava), amylose, amylopectin, glycogen, dextrans, celluloses, and derivatives thereof (e.g., methylcelluloses, hydroxyalkylcelluloses, hydroxyethylcelluloses, and carboxymethylcelluloses), mannans, xylans, lignins, arabans, galactans, galacturonans, chitins, chitosans, glucuronoxylans, arabinoxylans, xyloglucans, glucomannans, pectic acids, and pectins, alginic acid and alginates, arabinogalactans, carrageenans, agars, glycosaminoglucans, gum arabics, gum tragacanths, ghatti gums, karaya gums, carob gums, galactomannans, such as guar gums, and nonionic derivatives thereof (e.g., hydroxypropyl guar), sclerotium gum and xanthan gums, and mixtures thereof.

For example, the polysaccharides that may be used are chosen from those described, for example, in “Encyclopedia of Chemical Technology”, Kirk-Othmer, Third Edition, 1982, Volume 3, pp. 896-900, and Volume 15, pp. 439-458, in “Polymers in Nature” by E. A. MacGregor and C. T. Greenwood, published by John Wiley & Sons, Chapter 6, pp. 240-328, 1980, and in “Industrial Gums-Polysaccharides and their Derivatives”, edited by Roy L. Whistler, Second Edition, published by Academic Press Inc., the content of these three publications being entirely incorporated by reference.

For example, starches, guar gums, celluloses, and derivatives thereof can be used.

Among the starches that may be used, mention may be made, for example, of macromolecules in the form of polymers comprising base units which are anhydroglucose units. The number of these units and their assembly make it possible to distinguish between amylose (linear polymer) and amylopectin (branched polymer). The relative proportions of amylose and amylopectin, as well as their degree of polymerization, can vary according to the botanical origin of the starches.

The molecules of starches used may have cereals or tubers as their botanical origin. Thus, the starches can be, for example, chosen from maize, rice, cassava, tapioca, barley, potato, wheat, sorghum, and pea starches.

Starches generally exist in the form of a white powder, insoluble in cold water, whose elementary particle size ranges from 3 to 100 microns.

The starches may be optionally C₁-C₆ hydroxyalkylated or C₁-C₆ acylated (such as acetylated). The starches may have also undergone heat treatments.

Distarch phosphates or compounds rich in distarch phosphate, such as the products provided under the references PREJEL VA-70-T AGGL (gelatinized hydroxypropylated cassava distarch phosphate) or PREJEL TK1 (gelatinized cassava distarch phosphate) or PREJEL 200 (gelatinized acetylated cassava distarch phosphate) by the company AVEBE, may also be used.

The guar gums can be modified or unmodified.

The unmodified guar gums are, for example, the products sold under the name Vidogum GH 175 by the company Unipectine and under the names Meypro-Guar 50 and Jaguar C by the company Meyhall.

The modified nonionic guar gums are, for example, modified with C₁-C₆ hydroxyalkyl groups.

Among the hydroxyalkyl groups that may be mentioned are, for example, hydroxymethyl, hydroxyethyl, hydroxypropyl, and hydroxybutyl groups.

These guar gums are well known in the prior art and can be prepared, for example, by reacting the corresponding alkene oxides such as propylene oxides, with the guar gum so as to obtain a guar gum modified with hydroxypropyl groups.

The degree of hydroxyalkylation, which corresponds to the number of alkylene oxide molecules consumed by the number of free hydroxyl functions present on the guar gum, may, for example, range from 0.4 to 1.2.

Such nonionic guar gums optionally modified with hydroxyalkyl groups are sold, for example, under the trade names Jaguar HP8, Jaguar HP60, Jaguar HP120, Jaguar DC 293, and Jaguar HP 105 by the company Rhodia Chimie (Meyhall) or under the name Galactasol 4H_{4FD2} by the company Aqualon.

Among the celluloses and cellulose derivatives, such as cellulose modified with hydroxyalkyl groups, that are used are, for example, hydroxypropylmethylcellulose, hydroxyethylcellulose, and hydroxypropylcellulose, as well as hydrophobicized hydroxypropylmethylcellulose. Mention may be made of the products sold under the names Klucel E F, Klucel H, Klucel L H F, Klucel M F, and Klucel G by the company Aqualon.

(vii) The fatty alcohols are, for example, chosen from myristyl alcohol, cetyl alcohol, stearyl alcohol, and behenyl alcohol.

In addition, the (e) thickener may be selected from (viii) AMPS (co)polymers.

The AMPS (co)polymer means a water-soluble or water-dispersible, crosslinked or non-crosslinked, homopolymer of acrylamido-2-methylpropanesulfonic acid (AMPS) monomer, or copolymer of AMPS monomer and at least one comonomer.

Thus, the AMPS (co)polymer may be a water-soluble or water-dispersible, crosslinked or non-crosslinked polymer comprising, at least, acrylamido-2-methylpropanesulfonic acid (AMPS) monomer.

The AMPS (co)polymer may be preferably totally neutralized or virtually totally neutralized, i.e., at least 90% neutralized.

The AMPS (co)polymers may be crosslinked or non-crosslinked.

When the AMPS polymers are crosslinked, the crosslinking agents may be selected from among the polyolefinically unsaturated compounds commonly used for the crosslinking of polymers obtained by free-radical polymerization.

Examples of crosslinking agents that may be mentioned include divinylbenzene, diallyl ether, dipropylene glycol diallyl ether, polyglycol diallyl ethers, triethylene glycol divinyl ether, hydroquinone diallyl ether, ethylene glycol or tetraethylene glycol di(meth)acrylate, trimethylolpropane triacrylate, methylenebisacrylamide, methylenebismethacrylamide, triallylamine, triallyl cyanurate, diallyl maleate, tetraallylethylenediamine, tetraallyloxyethane, trimethylolpropane diallyl ether, allyl (meth)acrylate, allylic ethers of alcohols of the sugar series, or other allylic or vinyl ethers of polyfunctional alcohols, and also allylic esters of phosphoric and/or vinylphosphonic acid derivatives, or mixtures of these compounds.

According to one embodiment of the present invention, the crosslinking agent is selected from among methylenebis-acrylamide, allyl methacrylate and trimethylolpropane triacrylate (TMPTA). The degree of crosslinking generally ranges from 0.01 mol % to 10 mol % and more particularly from 0.2 mol % to 2 mol % relative to the polymer.

The AMPS polymers in accordance with the present invention are water-soluble or water-dispersible. In this case they are:
either
“homopolymers” polymerized from only AMPS monomers and, if they are crosslinked, one or more crosslinking agents such as those defined above;
or
“copolymers” polymerized from AMPS and from one or more hydrophilic or hydrophobic ethylenically unsaturated monomers and, if they are crosslinked, one or more crosslinking agents such as those defined above.

When the said copolymers are polymerized from hydrophobic ethylenically unsaturated monomers, these monomers may comprise no fatty chain and are preferably present in small amounts. For the purposes of the present invention, the term “fatty chain” means any hydrocarbon-based chain containing at least 7 carbon atoms.

Thus, the AMPS polymer may be selected from the group consisting of:
(i) crosslinked or non-crosslinked AMPS homopolymers;
(ii) crosslinked or non-crosslinked copolymers obtained from AMPS and from one or more hydrophilic ethylenically unsaturated monomers or hydrophobic ethylenically unsaturated monomers not containing a fatty chain; and
(iii) mixtures thereof.

The term “water-soluble or water-dispersible” means polymers which, when introduced into an aqueous phase at 25° C., to a mass concentration equal to 1%, make it possible to obtain a macroscopically homogeneous and transparent solution, i.e., a solution that has a maximum light transmittance value, at a wavelength equal to 500 nm, through a sample 1 cm thick, of at least 60% and preferably of at least 70%.

The “homopolymers” according to the present invention are preferably crosslinked and neutralized, and they may be prepared according to a preparation process comprising the following steps:
(a) the monomer such as AMPS in free form is dispersed or dissolved in a solution of tert-butanol or of water and tert-butanol;
(b) the solution or dispersion of the monomer obtained in (a) is neutralized with one or more mineral or organic bases, preferably ammonia NH₃, in an amount making it possible to obtain a degree of neutralization of the sulfonic acid functions of the polymer ranging from 90% to 100%;
(c) the crosslinking monomer(s) is(are) added to the solution or dispersion obtained in (b); and
(d) a standard free-radical polymerization is performed in the presence of free-radical initiators at a temperature ranging from 10 to 150° C.; the polymer precipitates in the solution or dispersion based on tert-butanol.

The water-soluble or water-dispersible AMPS copolymers according to the present invention may be polymerized from water-soluble ethylenically unsaturated monomers, hydrophobic monomers or mixtures thereof.

The water-soluble comonomers may be ionic or nonionic.

Among the ionic water-soluble comonomers, examples that may be mentioned include the following compounds and the salts thereof:
(meth)acrylic acid,
styrenesulfonic acid,
vinylsulfonic acid and (meth)allylsulfonic acid,
vinylphosphonic acid,
maleic acid,
itaconic acid,
crotonic acid,
the water-soluble vinyl monomers of formula (A) below:

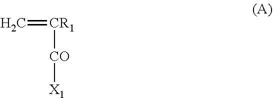

in which:
R₁ is H, —CH₃, —C₂H₅ or —C₃H₇;
X₁ is selected from among:
alkyl ethers of —OR₂ type in which R₂ is a linear or branched, saturated or unsaturated hydrocarbon-based radical containing from 1 to 6 carbon atoms, substituted with at least one sulfonic (—SO₃—) and/or sulfate (—SO₄—) and/or phosphate (—PO₄H₂—) group.

Among the nonionic water-soluble comonomers, examples that may be mentioned include:
(meth)acrylamide,
N-vinylacetamide and N-methyl-N-vinylacetamide,
N-vinylformamide and N-methyl-N-vinylformamide,
maleic anhydride,
vinylamine,
N-vinyllactams comprising a cyclic alkyl group containing 4 to 9 carbon atoms, such as n-vinylpyrrolidone, N-butyrolactam and N-vinylcaprolactam,
vinyl alcohol of formula CH₂=CHOH,
the water-soluble vinyl monomers of formula (B) below:

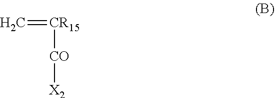

in which:
R₁₅ is H, —CH₃, —C₂H₅ or —C₃H₇;
X₂ is selected from among:
alkyl ethers of —OR₁₆ type in which R₁₆ is a linear or branched, saturated or unsaturated hydrocarbon-based radical containing from 1 to 6 carbons, optionally substituted with a halogen atom (iodine, bromine, chlorine or fluorine); a hydroxyl group (—OH); ether.

Mention is made, for example, of glycidyl (meth)acrylate, hydroxyethyl methacrylate and (meth)acrylates of ethylene glycol, of diethylene glycol or of polyalkylene glycol.

Among the fatty-chain-free hydrophobic comonomers, examples that may be mentioned include: styrene and its derivatives, such as 4-butylstyrene, α-methylstyrene and vinyltoluene,
vinyl acetate of formula CH₂═CH—OCOCH₃;
vinyl ethers of formula CH₂═CHOR in which R is a linear or branched, saturated or unsaturated hydrocarbon-based radical containing from 1 to 6 carbons;
acrylonitrile,
caprolactone,
vinyl chloride and vinylidene chloride,
silicone derivatives, which provide silicone polymers after polymerization, such as methacryloxypropyltris(trimethylsiloxy)silane and silicone methacrylamides,
the hydrophobic vinyl monomers of formula (C) below:

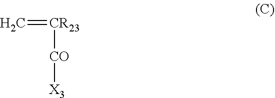

in which:
R₂₃ is H, —CH₃, —C₂H₅ or —C₃H₇;
X₃ is selected from among:
alkyl ethers of —OR₂₄ type in which R₂₄ is a linear or branched, saturated or unsaturated hydrocarbon-based radical containing from 1 to 6 carbon atoms.

Mention is made, for example, of methyl methacrylate, ethyl methacrylate, n-butyl (meth)acrylate, tert-butyl (meth)acrylate, cyclohexyl acrylate, isobornyl acrylate, and 2-ethylhexyl acrylate.

The water-soluble or water-dispersible AMPS polymers of the present invention preferably have a molar mass ranging from 50,000 g/mol to 10,000,000 g/mol, preferably from 80,000 g/mol to 8,000,000 g/mol and even more preferably from 100,000 g/mol to 7,000,000 g/mol.

Examples of water-soluble or water-dispersible AMPS homopolymers in accordance with the present invention that may be mentioned include crosslinked or non-crosslinked polymers of sodium acrylamido-2-methylpropanesulfonate, such as the polymer in the commercial product Simulgel 800 (INCI name: Sodium Polyacryloyldimethyltaurate).

Examples of water-soluble or water-dispersible AMPS copolymers in accordance with the present invention that may be mentioned include:
acrylamide/sodium acrylamido-2-methylpropanesulfonate crosslinked copolymers, such as the copolymer in the commercial product Sepigel 305 (INCI name: Polyacrylamide/C₁₃-C₁₄ Isoparaffin/Laureth-7) or the copolymer in the commercial product sold under the trademark Simulgel 600 (INCI name: Acrylamide/Sodium Acryloyldimethyltaurate/Isohexadecane/Polysorbate-80) by SEPPIC;
copolymers of AMPS and of vinylpyrrolidone or of vinylformamide, such as the copolymer in the commercial product sold under the name Aristoflex AVC by Clariant (INCI name: Ammonium Acryloyldimethyltaurate/V—P Copolymer) but neutralized with sodium hydroxide or potassium hydroxide;
copolymers of AMPS and of sodium acrylate, for instance AMPS/sodium acrylate copolymer such as the copolymer in the commercial product sold under the name Simulgel EG by SEPPIC (INCI name: Sodium Acrylate/Sodium Acryloyldimethyltaurate Copolymer (and) Isohexadecane (and) Polysorbate-80); and
copolymers of AMPS and of hydroxyethyl acrylate, for instance AMPS/hydroxyethyl acrylate copolymer, such as the copolymer in the commercial product sold under the name Simulgel NS by SEPPIC (INCI name: Hydroxyethyl acrylate/Sodium Acryloyldimethyltaurate copolymer (and) Squalane (and) Polysorbate-60).

Thus, it may be preferable that the AMPS polymer be selected from the group consisting of: an acrylamide/sodium acrylamido-2-methylpropanesulfonate (acrylamide/sodium acryloyldimethyltaurate) crosslinked copolymer;
a copolymer of AMPS and of vinylpyrrolidone or of vinylformamide;
a copolymer of AMPS and of sodium acrylate (sodium acrylate/sodium acryloyldimethyltaurate copolymer);
a copolymer of AMPS and of hydroxyethyl acrylate; and
a mixture thereof.

When AMPS monomers are copolymerized with ethylenically unsaturated hydrophobic monomers containing a fatty chain (e.g., C₆-C₅₀ chain), the polymer obtained is amphiphilic, that is, it contains both a hydrophilic part and a hydrophobic part.

The amphiphilic AMPS copolymers may contain, in addition, one or more ethylenically unsaturated monomers containing no fatty chain, such as (meth)acrylic acids, their β-substituted alkyl derivatives or their esters obtained with monoalcohols or mono- or polyalkylene glycols, (meth)acrylamides, vinylpyrrolidone, vinylformamide, maleic anhydride, itaconic acid or maleic acid or mixtures of these compounds.

Suitable amphiphilic polymers include, for example, those obtained from AMPS and from at least one ethylenically unsaturated hydrophobic monomer containing at least one hydrophobic part having from 6 to 50 carbon atoms, preferably from 6 to 22 carbon atoms, more preferably from 6 to 18 carbon atoms and most particularly 12 to 18 carbon atoms.

Examples of these copolymers are described in particular in patent application EP-A-750899, U.S. Pat. No. 5,089,578 and in the following publications by Yotaro Morishima:
“Self-assembling amphiphilic polyelectrolytes and their nanostructures—Chinese Journal of Polymer Science Vol. 18, No. 40, (2000), 323-336”;
“Miscelle formation of random copolymers of sodium 2-(acrylamido)-2-methylpropanesulfonate and a non-ionic surfactant macromonomer in water as studied by fluorescence and dynamic light scattering—Macromolecules 2000, Vol. 33, No. 10-3694-3704”;
“Solution properties of miscelle networks formed by non-ionic moieties covalently bound to a polyelectrolyte: salt effects on rheological behavior—Langmuir, 2000, Vol. 16, No. 12, 5324-5332”;
“Stimuli responsive amphiphilic copolymers of sodium 2-(acrylamido)-2-methylpropanesulfonate and associative macromonomers—Polym. Preprint, Div. Polym. Chem. 1999, 40(2), 220-221”.

The ethylenically unsaturated hydrophobic monomers of these particular copolymers are preferably chosen from acrylates, alkylacrylates, acrylamides and alkylacrylamides of the following formula (III):

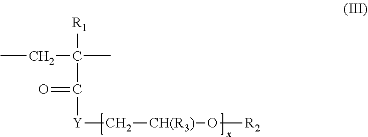

in which R₁ and R₃, which are identical or different, denote a hydrogen atom or a substantially linear or branched C₁-C₆ alkyl radical (preferably methyl); Y denotes O or NH; R₂ denotes a hydrophobic hydrocarbon radical containing at least from 6 to 50 carbon atoms, preferably from 6 to 22 carbon atoms, more preferably from 6 to 18 carbon atoms and most preferably from 12 to 18 carbon atoms; x denotes a number of moles of alkylene oxide and preferably varies from 0 to 100.

The radical R₂ is preferably chosen from substantially linear C₆-C₁₈ alkyl radicals (for example n-hexyl, n-octyl, n-decyl, n-hexadecyl, n-dodecyl, n-octadecyl) or branched or cyclic C₆-C₁₈ alkyl radicals (for example cyclododecane (C₁₂) or adamantane (C₁₀)) perfluorinated C₆-C₁₈ alkyl radicals (for example the group of formula —(CH₂)₂—(CF₂)₉—CF₃); the cholesteryl radical (C₂₇) or a cholesterol ester residue such as the cholesteryl oxyhexanoate group; polycyclic aromatic groups such as naphthalene or pyrene. Among these radicals, the most preferred are the substantially linear alkyl radicals, preferably the n-dodecyl, n-hexadecyl or n-octadecyl radical, and mixtures thereof.

According to a particularly preferred embodiment of the present invention, the monomer of formula (III) contains at least one alkylene oxide unit (x≥1), preferably several alkylene oxide units (x>1) constituting a polyoxyalkylenated chain. The polyoxyalkylenated chain preferably consists of ethylene oxide units and/or propylene oxide units, more preferably consists of ethylene oxide units. The number of oxyalkylenated units (or number of moles of alkylene oxide) preferably varies from 3 to 100, more preferably from 3 to 50 and most preferably from 7 to 25.

Suitable polymers include, for example,
crosslinked or non-crosslinked and neutralized or normeutralized copolymers containing from 15 to 60% by weight of AMPS units and from 40 to 85% by weight of (C₈-C₁₆)alkyl(meth)acrylamide units or (C₈-C₁₆)alkyl(meth)acrylate units, relative to the polymer, such as those described in application EP-A-750 899; and
terpolymers containing from 10 to 90 mol % of acrylamide units, from 0.1 to 10 mol % of AMPS units and from 5 to 80 mol % of n-(C₆-C₁₈)alkylacrylamide units, relative to the polymer, such as those described in U.S. Pat. No. 5,089,578.

Suitable amphiphilic polymers include, for example,
copolymers of completely neutralized AMPS and of n-dodecyl, n-hexadecyl and/or n-octadecyl methacrylate, and
copolymers of AMPS and of n-dodecylmethacrylamide, which are non-crosslinked and crosslinked, such as those described in the articles by Morishima cited above.

Preferably, the copolymers consist of 2-acrylamido-2-methylpropanesulphonic acid (AMPS) units of the following formula (IV):

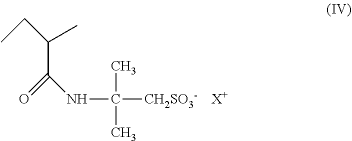

in which X⁺ is a proton, an alkali metal cation, an alkaline-earth metal cation or the ammonium ion, and of units of the following formula (V):

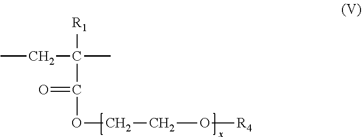

in which x denotes an integer ranging from 3 to 100, preferably from 3 to 50, more preferably from 7 to 25; R₁ has the same meaning as that indicated above in formula (I) and R₄ denotes a substantially linear or branched C₆-C₂₂, more preferably C₁₀-C₂₂, alkyl.

The particularly preferred amphiphilic polymers of this type are those for which x=25, R₁ denotes methyl and R₄ represents n-dodecyl; they are described in the articles by Morishima mentioned above. Other preferred polymers are those for which x=8 or 25, R₁ denotes methyl and R₄ represents n-hexadecyl (C₁₆), n-octadecyl (C₁₈) or n-dodecyl (C₁₂), or mixtures thereof.

The polymers for which X⁺ denotes sodium or ammonium are most preferred.

The preferred amphiphilic polymers used in the composition in accordance with the present invention may be obtained according to conventional free-radical polymerization methods in the presence of one or more initiators such as, for example, azobisisobutyronitrile (AIBN), azobisdimethylvaleronitrile, 2,2-azobis[2-amidinopropane]hydrochloride (ABAH), organic peroxides such as dilauryl peroxide, benzoyl peroxide, tert-butyl hydroperoxide, and the like, inorganic peroxidized compounds such as potassium or ammonium persulphate or H₂O₂, optionally in the presence of reducing agents.

These amphiphilic polymers are preferably obtained by free-radical polymerization in tert-butanol medium from which they precipitate. Using polymerization by precipitation from tert-butanol, it is possible to obtain a size distribution of the polymer particles which is particularly favorable.

The reaction may be carried out at a temperature of between 0 and 150° C., preferably between 1° and 100° C., either at atmospheric pressure, or under reduced pressure. It may also be carried out under an inert atmosphere, preferably under nitrogen.

According to this method, 2-acrylamido-2-methylpropanesulphonic acid (AMPS) or one of its sodium or ammonium salts can be polymerized with an ester of (meth)acrylic acid and
of a C₁₀-C₁₈ alcohol oxyethylenated with 8 mol of ethylene oxide (GENAPOL® C-080 from the company CLARIANT),
of a C₁₁ oxo alcohol oxyethylenated with 8 mol of ethylene oxide (GENAPOL® UD-080 from the company CLARIANT),
of a C₁₁ oxo alcohol oxyethylenated with 7 mol of ethylene oxide (GENAPOL® UD-070 from the company CLARIANT),
of a C₁₂-C₁₄ alcohol oxyethylenated with 7 mol of ethylene oxide (GENAPOL® LA-070 from the company CLARIANT),
of a C₁₂-C₁₄ alcohol oxyethylenated with 9 mol of ethylene oxide (GENAPOL® LA-090 from the company CLARIANT),
of a C₁₂-C₁₄ alcohol oxyethylenated with 11 mol of ethylene oxide (GENAPOL® LA-110 from the company CLARIANT),
of a C₁₆-C₁₈ alcohol oxyethylenated with 8 mol of ethylene oxide (GENAPOL® T-080 from the company CLARIANT),
of a C₁₆-C₁₈ alcohol oxyethylenated with 15 mol of ethylene oxide (GENAPOL® T-150 from the company CLARIANT),
of a C₁₆-C₁₈ alcohol oxyethylenated with 11 mol of ethylene oxide (GENAPOL® T-110 from the company CLARIANT),
of a C₁₆-C₁₈ alcohol oxyethylenated with 20 mol of ethylene oxide (GENAPOL® T-200 from the company CLARIANT),
of a C₁₆-C₁₈ alcohol oxyethylenated with 25 mol of ethylene oxide (GENAPOL® T-250 from the company CLARIANT), or
of a C₁₈-C₂₂ alcohol oxyethylenated with 25 mol of ethylene oxide and/or of a C₁₆-C₁₈ iso-alcohol oxyethylenated with 25 mol of ethylene oxide.

The mol % concentration of the units of formula (IV) and of the units of formula (V) in the polymers according to the present invention vary according to the desired cosmetic application and the rheological properties sought for the formulation. It may vary between 0.1 and 99.9 mol %.

Preferably, for the most hydrophobic polymers, the molar proportion of units of formula (IV) or (V) varies from 50.1 to 99.9%, more preferably from 70 to 95%, and most preferably from 80 to 90%.

Preferably, for the polymers which are not very hydrophobic, the molar proportion of units of formula (IV) or (V) varies from 0.1 to 50%, more preferably from 5 to 25%, and most preferably from 10 to 20%.

The distribution of the monomers in the polymers of the present invention may be, for example, alternating, block (including multiblock) or random.

Mention may in particular be made, as hydrophobic modified polymers of this type, of the copolymer of AMPS and of ethoxylated C₁₂-C₁₄ alkyl methacrylate (noncrosslinked copolymer obtained from Genapol LA-070 and from AMPS) (CTFA name: Ammonium Acryloyldimethyltaurate/Laureth-7 Methacrylate Copolymer) sold under the name Aristoflex LNC by Clariant, and the copolymer of AMPS and of ethoxylated (25 EO) stearyl methacrylate (copolymer crosslinked with triacrylate obtained from Genapol trimethylolpropane T-250 and from AMPS) (CTFA name: Ammonium Acryloyldimethyltaurate/Steareth-25 Methacrylate Crosspolymer) sold under the name Aristoflex HMS by Clariant.

It is preferable that the (e) thickener be selected from polysaccharide, AMPS (co)polymer, and a mixture thereof.

It is more preferable that the (e) thickener be selected from xanthan gum, amphiphilic AMPS copolymers such as Ammonium Acryloyldimethyltaurate/Steareth-25 Methacrylate Crosspolymer, and a mixture thereof.

The amount of the (e) thickener(s) in the composition according to the present invention may be 0.01% by weight or more, preferably 0.05% by weight or more, and more preferably 0.1% by weight or more, relative to the total weight of the composition.

The amount of the (e) thickener(s) in the composition according to the present invention may be 10% by weight or less, preferably 5% by weight or less, and more preferably 1% by weight or less, relative to the total weight of the composition.

The amount of the (e) thickener(s) in the composition according to the present invention may be from 0.01% to 10% by weight, preferably from 0.05% to 5% by weight, and more preferably from 0.1% to 1% by weight, relative to the total weight of the composition.

### [Powder]

The composition according to the present invention comprises (f) at least one powder. Two or more powders may be used in combination. Thus, a single type of powder or a combination of different types of powders may be used.

According to the present invention, the (f) powder is insoluble in a physiologically acceptable volatile medium such as water.

For the purposes of the present invention, the term “insoluble” powder means a powder with a solubility in a physiologically acceptable volatile medium such as water at 25° C. of less than 1% by weight, preferably less than 0.1% by weight and more preferably less than 0.01% by weight, relative to the total weight of the powder, and most preferably with no solubility.

The (f) powder is in the form of a particle or particles.

The diameter of the (f) powder is not limited. The average particle size of the (f) powder is preferably 10 nm or more, more preferably 50 nm or more, and even more preferably 100 nm or more, and is preferably 1,000 μm or less, more preferably 500 μm or less, and even more preferably 300 μm or less. Thus, the (f) powder may have an average particle size of from 10 nm to 1,000 μm, preferably from 50 nm to 500 μm, and more preferably from 100 nm to 300 μm.

It is preferable that the average particle size of the (f) powder be 0.1 μm or more, more preferably 0.3 μm or more, and even more preferably 0.5 μm or more.

It is preferable that the average particle size of the (f) powder be 15 μm or less, more preferably 12 μm or less, and even more preferably 10 μm or less.

The average particle size may be a number-average particle size which can be measured by dynamic light scattering with, for example, Nicomp Z380.

The (f) powder is preferably in the form of a solid.

The form of the (f) powder is not limited. The (f) powder may be in the form of a sphere, a plate, and the like. The (f) powder may be hollow or porous.

The (f) powder may be selected from pigments, fillers, and mixtures thereof.

### (Pigment)

The term “pigments” should be understood as meaning white or colored and inorganic or organic particles which are insoluble in a physiologically acceptable volatile medium and which are intended to color and/or opacify the resulting film.

The pigments preferably have an absorption ranging from 380 to 780 nm, and in at least one embodiment, an absorption with a maximum in this absorption range.

The pigments may be organic pigments. As used herein, the term “organic pigment” means any pigment that satisfies the definition in Ullmann's encyclopedia in the chapter on organic pigments.

The organic pigment may be chosen, for example, from nitroso, nitro, azo, xanthene, quinoline, anthraquinone, phthalocyanin, metal complex, isoindolinone, isoindoline, quinacridone, perinone, perylene, diketopyrrolopyrrole, thioindigo, dioxazine, triphenylmethane, and quinophthalone compounds.

The at least one organic pigment may be chosen from, for example, carmine, carbon black, aniline black, melanin, azo yellow, quinacridone, phthalocyanin blue, sorghum red, the blue pigments codified in the Color Index under the references CI 42090, 69800, 69825, 73000, 74100, and 74160, the yellow pigments codified in the Color Index under the references CI 11680, 11710, 15985, 19140, 20040, 21100, 21108, 47000, and 47005, the green pigments codified in the Color Index under the references CI 61565, 61570, and 74260, the orange pigments codified in the Color Index under the references CI 11725, 15510, 45370, and 71105, the red pigments codified in the Color Index under the references CI 12085, 12120, 12370, 12420, 12490, 14700, 15525, 15580, 15620, 15630, 15800, 15850, 15865, 15880, 17200, 26100, 45380, 45410, 58000, 73360, 73915, and 75470, and the pigments obtained by oxidative polymerization of indole or phenolic derivatives as described, for example, in French Patent No. 2 679 771.

These pigments may also be in the form of composite pigments as described, for example, in European Patent No. 1 184 426. These composite pigments may be composed, for instance, of particles comprising an inorganic nucleus at least partially coated with an organic pigment and at least one binder to fix the organic pigments to the nucleus.

Other examples include pigmentary pastes of organic pigments such as the products sold by the company Hoechst under the names: Jaune Cosmenyl IOG: Pigment Yellow 3 (CI 11710); Jaune Cosmenyl G: Pigment Yellow 1 (CI 11680); Orange Cosmenyl GR: Pigment Orange 43 (CI 71105); Rouge Cosmenyl R″: Pigment Red 4 (CI 12085); Carmine Cosmenyl FB: Pigment Red 5 (CI 12490); Violet Cosmenyl RL: Pigment Violet 23 (CI 51319); Bleu Cosmenyl A2R: Pigment Blue 15.1 (CI 74160); Vert Cosmenyl GG: Pigment Green 7 (CI 74260); and Noir Cosmenyl R: Pigment Black 7 (CI 77266).

The at least one pigment may also be chosen from lakes. As used herein, the term “lake” means insolubilized dyes adsorbed onto insoluble particles, the complex or the compound thus obtained remaining insoluble during use.

The inorganic substrates onto which the dyes are adsorbed may include, for example, alumina, silica, calcium sodium borosilicate, calcium aluminum borosilicate, and aluminum.

Non-limiting examples of organic dyes include cochineal carmine and the products known under the following names: D&C Red 21 (CI 45 380), D&C Orange 5 (CI 45 370), D&C Red 27 (CI 45 410), D&C Orange 10 (CI 45 425), D&C Red 3 (CI 45 430), D&C Red 4 (CI 15 510), D&C Red 33 (CI 17 200), D&C Yellow 5 (CI 19 140), D&C Yellow 6 (CI 15 985), D&C Green (CI 61 570), D&C Yellow 10 (CI 77 002), D&C Green 3 (CI 42 053), and D&C Blue 1 (CI 42 090).

An additional non-limiting example of a lake is the product known under the following name: D&C Red 7 (CI 15 850:1).

The at least one pigment may also be a pigment with special effects. As used herein, the term “pigments with special effects” means pigments that generally create a non-uniform colored appearance (characterized by a certain shade, a certain vivacity, and/or a certain lightness) that changes as a function of the conditions of observation (light, temperature, observation angle, etc.). They thus contrast with white or colored pigments that afford a standard uniform opaque, semi-transparent, or transparent shade.

Two types of pigment with special effects exist: those with a low refractive index, such as fluorescent, photochromic, and thermochromic pigments, and those with a high refractive index, such as nacres and flakes.

The at least one pigment may also be chosen from pigments with an interference effect that are not fixed onto a substrate, for instance, liquid crystals (Helicones HC from Wacker), and holographic interference flakes (Geometric Pigments or Spectra f/x from Spectratek).

The pigments with special effects may also comprise fluorescent pigments, whether these are substances that are fluorescent in daylight or that produce an ultraviolet fluorescence, phosphorescent pigments, photochromic pigments, and thermochromic pigments.

The pigment may also be an inorganic pigment, in a preferred embodiment. As used herein, the term “inorganic pigment” means any pigment that satisfies the definition in Ullmann's encyclopedia in the chapter on inorganic pigments. Preferably, the inorganic pigments comprise at least one inorganic material. Non-limiting examples of inorganic pigments that are useful in the present invention include metal oxides, in particular, transition metal oxides, such as zirconium oxides, cerium oxides, iron oxides, zinc oxides, chromium oxides, manganese violet, ultramarine blue, chromium hydrate, ferric blue, and titanium dioxide. The following inorganic pigments may also be used: Ta₂O₅, Ti₃O₅, Ti₂O₃, TiO, and ZrO₂ as a mixture with TiO₂, ZrO₂, Nb₂O₅, CeO₂, and ZnS.

The pigment may also be a pearlescent pigment.

The term “pearlescent pigments”, which may also be called “nacres” or “nacreous pigments”, should be understood as meaning colored particles of any form, which may or may not be iridescent, especially produced by certain molluscs in their shell, or alternatively synthesized, and which have a color effect via optical interference.

The pearlescent pigment may have a particle size of 15 μm or less, preferably 12 μm or less, and more preferably 10 μm or less and/or may have a colored reflection.

It may be preferable that the pearlescent pigment have a particle size of 1 μm or more, preferably 3 μm or more, and more preferably 5 μm or more.

The particle size here can be expressed as a mean volume diameter (D[0.5]).

The pearlescent pigment may have a reflection with a color such as white, blue, yellow, pink, red, bronze, orange, brown, gold and copper.

It may be preferable that the pearlescent pigment can show a white color. For example, the pearlescent pigment can show a white color on a white background.

It may also be preferable that the pearlescent pigment have a blue reflection. For example, the pearlescent pigment can show a blue reflection on a black background. Here, the term “blue” means visible light with a wavelength of from about 450 to about 495 nm.

The pearlescent pigment with a blue reflection can contribute anti-yellowness. Therefore, it can be advantageous in covering color imperfections on a keratin substance, such as spots on skin.

The pearlescent pigment with a limited particle size and a colored reflection can contribute to color correction of a keratin substance such as skin by mixing the colored reflection with the original color of the keratin substance, and to natural appearance. It should be noted that the use of conventional pearlescent pigments with a particle size of more than 15 μm may not be able to sufficiently cover the keratin substance, and cannot provide sufficient brightness but provides a glittering appearance and unnatural finish.

The pearlescent pigment can be in any shape. For example, it is possible to use a pearlescent pigment in the form of a plate with an aspect ratio of at least 5, preferably more than 10, more preferably more than 20, and even more preferably more than 50. The aspect ratio can be determined by the average thickness and the average length according to the formula: aspect ratio=length/thickness.

If a plate-like particle is used for the pearlescent pigment, it is preferable that the plate-like particle have a length of 15 μm or less, preferably 12 μm or less, and more preferably 10 μm or less.

Examples of the pearlescent pigment that may be mentioned include nacres such as a mica coated with titanium (oxide) or with bismuth oxychloride, and a mica coated with titanium (oxide) and with iron oxide(s), a mica coated with titanium (oxide) and chromium oxide(s), and mixtures thereof. They may also be mica particles at the surface of which are superposed at least two successive layers of metal oxides and/or of organic dyestuffs.

It may be preferable that the pearlescent pigment comprise mica and metal oxide, preferably titanium oxide and/or tin oxide.

Still, as examples of the pearlescent pigment, mention may also be made of particles comprising a borosilicate substrate coated with titanium oxide.

### (Filler)

The term “filler” should be understood as meaning an uncolored particle that is solid at room temperature and atmospheric pressure, and insoluble in a physiologically acceptable volatile medium, even when these ingredients are brought to a temperature above room temperature. Of inorganic or organic nature thereof, fillers make it possible to confer firmness on the composition according to the present invention and/or softness and uniformity on the make-up which may be formed by the composition.

The filler may be chosen from mineral and organic fillers. When the fillers are organic fillers, they are polymeric organic fillers. The filler may be particles of any form, for example, platelet-shaped, spherical, and oblong, irrespective of their crystallographic form (for example lamellar, cubic, hexagonal, and orthorhombic).

The fillers that may be used in the composition according to the present invention can be made from various inorganic and/or organic materials, and may include, but are not limited to, titanium dioxide; talc; natural or synthetic mica; alumina; aluminosilicate; calcium sodium borosilicate; calcium aluminum borosilicate; silica (or silicon dioxides); kaolin or other insoluble silicates such as clays; polyamides (Nylon®), poly-β-alanine and polyethylene powders; tetrafluoroethylene polymer (Teflon®) powders, powder starch; boron nitride; acrylic acid polymer powders; silicone resin microbeads, for instance “Tospearls®” from the company Toshiba; bismuth oxychlorides; precipitated calcium carbonate; magnesium carbonate and magnesium hydrogen carbonate; hydroxyapatite; hollow silica microspheres such as “Silica Beads SB 700®” and “Silica Beads SB 700®” from the company Maprecos, “Sunspheres H-33®” and “Sunspheres H-51®” from the company Asahi Glass; acrylic polymer microspheres such as those made from crosslinked acrylate copolymer “Polytrap 6603®” from the company R.P. Scherrer and those made from polymethyl methacrylate “Micropearl M100®” from the company SEPPIC; polyurea powders; polyurethane powders such as the hexamethylene diisocyanate and trimethylol hexyl lactone copolymer powder sold under the name “Plastic Powder D-400®” by the company Toshiki; glass or ceramic microcapsules; microcapsules of methyl acrylate or methacrylate polymers or copolymers, or alternatively, vinylidene chloride and acrylonitrile copolymers, for instance, “Expancel®” from the company Expancel; elastomeric crosslinked organopolysiloxane powders such as those sold under the name “KSP100®” by the company Shinetsu Chemical; porous cellulose beads such as those sold under the name of Cellulose Beads USF® by the company Daito Kasei; and mixtures thereof.

Among the silicas that are useful in the composition of the present invention, mention may be made of crystalline, microcrystalline and non-crystalline silicas.

By way of example, crystalline silicas that may be mentioned include quartz, tridymite, cristobalite, keatite, coesite and stishovite. The microcrystalline silicas are, for example, diatomite.

Among the non-crystalline forms that may be used are vitreous silica and other types of amorphous silicas such as colloidal silicas, silica gels, precipitated silicas and fumed silicas, for instance aerosils, and pyrogenic silicas. Porous silica such as an aerogel (silica silylate) is preferable.

In one embodiment of the present invention, the powder may comprise at least one inorganic material selected from the group consisting of mica, synthetic mica, talc, sericite, boron nitride, glass, calcium carbonate, barium sulfate, titanium oxide, hydroxyapatite, silica, silicate, calcium sodium borosilicate, zinc oxide, magnesium sulfate, magnesium carbonate, magnesium trisilicate, aluminum oxide, aluminum silicate, calcium silicate, calcium phosphate, magnesium oxide, bismuth oxychloride, kaolin, hydrotalcite, mineral clay, synthetic clay, iron oxide, and mixtures thereof.

In another embodiment of the present invention, the powder may comprise at least one organic material selected from the group consisting of polyurea, melamine-formaldehyde condensate, urea-formaldehyde condensate, aminoplast, polyurethane, polyacrylate, polyphosphate, polystyrene, polyester, polyamide, polyolefin, polysaccharide, silicone, silicone resin, protein, modified cellulose, and gum.

It is preferable that the (f) powder comprise at least one selected from the group consisting of boron nitride, glass (e.g., sodium borosilicate, calcium sodium borosilicate, calcium aluminum borosilicate), titanium dioxide (and) mica, and a mixture thereof.

If the (f) powder comprises boron nitride, it may have a refractive index of 1.6 or more, preferably between 1.6 and 2.2. In a preferred embodiment, the (f) powder comprising boron nitride may have an average particle size between 1 μm and 10 μm, and in particular between 1 μm and 6 μm.

As examples of commercial products of boron nitride, we may cite the following products: PUHP3008 from Saint Gobains Ceramics (mean particle size 6 μm), the PUHP1030L from Saint Gobains Ceramics (mean particle size 3 μm), the Softouch BN CC6058 powder from Momentive Performance Materials (mean particle size 5-15 μm), or mixtures thereof.

If the (f) powder comprises glass such as sodium borosilicate, calcium sodium borosilicate, calcium aluminum borosilicate, it may be in the form of a microsphere (hollow particle).

Examples of commercial products of glass microspheres useful in the present invention include hollow microspheres of calcium aluminum borosilicate (commercially available from Presperse Inc. under the trade name LUXSIL®), sodium borosilicate particulates (commercially available from PQ Corporation under the trade name Q-CEL 570), and calcium/sodium borosilicate hollow microspheres (commercially available from 3M under the trade names ES 22 and 1K), calcium/sodium borosilicate microspheres (commercially available from 3M under the trade name Scotchlite™ K20 product).

If the (f) powder comprises titanium dioxide (and) mica, it may be a nacreous pigment (nacres) or a pearlescent pigment. In other words, the titanium dioxide (and) mica may be a mica coated with, at least, titanium dioxide.

As examples of the titanium dioxide (and) mica, mention may be made of Ronaflair Balance Red/Blue/Gold/Green, and Timiron Silk Blue/Green/Red/Gold sold by the company Merck, Sunshine Soft Fine Gold/Red/Violet/Blue/Green sold by the company Sun Chemical, Helios R10Y/R/B/G sold by the company Topy, and Flamenco Summit Aqua series such as FLAMENCO SUMMIT RED R30D, FLAMENCO SUMMIT BLUE B30DC sold by the company BASF.

The amount of the (f) powder(s) in the composition according to the present invention may be 0.1% by weight or more, preferably 0.5% by weight or more, and more preferably 1% by weight or more, relative to the total weight of the composition.

The amount of the (f) powder(s) in the composition according to the present invention may be 15% by weight or less, preferably 10% by weight or less, and more preferably 5% by weight or less, relative to the total weight of the composition.

The amount of the (f) powder(s) in the composition according to the present invention may be from 0.1% to 15% by weight, preferably from 0.5% to 10% by weight, and more preferably from 1% to 5% by weight, relative to the total weight of the composition.

### [Water]

The composition according to the present invention comprises (g) water.

The amount of (g) water in the composition according to the present invention may be 45% by weight or more, preferably 50% by weight or more, and more preferably 55% by weight or more, relative to the total weight of the composition.

On the other hand, the amount of (g) water in the composition according to the present invention may be 90% by weight or less, preferably 85% by weight or less, and more preferably 80% by weight or less, relative to the total weight of the composition.

The amount of (g) water in the composition according to the present invention may range from 45% to 90% by weight, preferably from 50% to 85% by weight, more preferably from 55% to 80% by weight, relative to the total weight of the composition.

### [Vitamin B3 and Derivatives]

The composition according to the present invention may comprise (h) at least one compound selected from Vitamin B3 and derivatives thereof. A single type of the (h) compound may be used, but two or more different types of the (h) compounds may be used in combination.

Vitamin B3, also called vitamin PP, is a compound of the following formula (I):

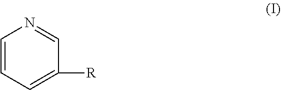

in which R may be —CONH₂ (niacinamide), —COOH (nicotinic acid or niacin), or CH₂OH (nicotinyl alcohol), —CO—NH—CH₂—COOH (nicotinuric acid) or —CO—NH—OH (niconityl hydroxamic acid). Niacinamide is preferable.

Vitamin B3 derivatives that may be mentioned include, for example, nicotinic acid esters such as tocopherol nicotinate, amides derived from niacinamide by substitution of the hydrogen groups of —CONH₂, products from reaction with carboxylic acids and amino acids, esters of nicotinyl alcohol and of carboxylic acids such as acetic acid, salicyclic acid, glycolid acid or palmitic acid.

Mention may also be made of the following derivatives: 2-chloronicotinamide, 6-methylnicotinamide, 6-aminonicotinamide, N-methylnicotinamide, N,N-dimethylnicotinamide, N-(hydroxymethyl)nicotinamide, quinolinic acid imide, nicotinanilide, N-benzylnicotinamide, N-ethylnicotinamide, nifenazone, nicotinaldehyde, isonicotinic acid, methylisonicotinic acid, thionicotinamide, nialamide, 2-mercaptonicotinic acid, nicomol and niaprazine, methyl nicotinate and sodium nicotinate.

Other vitamin B3 derivatives that may also be mentioned include its inorganic salts, such as chlorides, bromides, iodides or carbonates, and its organic salts, such as the salts obtained by reaction with carboxylic acids, such as acetate, salicylate, glycolate, lactate, malate, citrate, mandelate, tartrate, etc.

The amount of the (h) compound(s) in the composition according to the present invention may be 1% by weight or more, preferably 2% by weight or more, and more preferably 3% by weight or more, relative to the total weight of the composition.

On the other hand, the amount of the (h) compound(s) in the composition according to the present invention may be 20% by weight or less, preferably 15% by weight or less, and more preferably 10% by weight or less, relative to the total weight of the composition.

The amount of the (h) compound(s) in the composition according to the present invention may range from 1% to 20% by weight, preferably from 2% to 15% by weight, more preferably from 3% to 10% by weight, relative to the total weight of the composition.

### [Gemini Surfactant]

The composition according to the present invention may comprise (i) at least one gemini surfactant. A single type of gemini surfactant may be used, but two or more different types of gemini surfactants may be used in combination.

The (i) gemini surfactant or dimeric surfactants used in the present invention are well known. For a detailed description of the various chemical structures and their physicochemical properties, reference may be made to the following publications:
Milton J. Rosen, Gemini Surfactants, Properties of surfactant molecules with two hydrophilic groups and two hydrophobic groups, Cosmetics & Toiletries magazine, Vol. 113, December 1998, pp. 49-55; and
Milton J. Rosen, Recent Developments in Gemini Surfactants, Allured's Cosmetics & Toiletries magazine, July 2001, Vol. 116, No. 7, pp. 67-70.

The (i) gemini surfactant may be selected from compounds with two or more fatty amide groups (hereafter, it may be referred to as “amide compound”). The amide compound may be represented by the following formula (A):

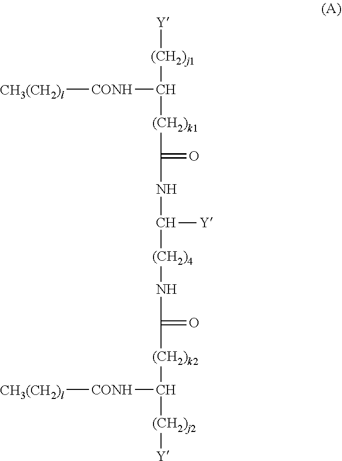

wherein
Y′ independently denotes a carboxylic acid group or an alkaline salt of a carboxylic acid group such as a sodium salt of a carboxylic acid group,
j1, k1, j2 and k2 denote an integer such that (j1, k1, j2, k2)=any of (2,0,2,0), (2,0,0,2), (0,2,2,0) and (0,2,0,2), and
1 denotes an integer from 6 to 16, preferably 8 to 14, and more preferably 10 to 12.

Preferably, in formula (A), L denotes an integer from 8 to 12, j1=j2=0, and k1=k2=2.

Most preferably, in formula (A), Y′ is —COONa, j1=j2=0, k1=k2=2; and L=10.

The amide compound can be prepared by, for example, reacting a long chain N-acyl acidic amino acid anhydride with a basic amino acid, such as lysine, in water and/or a mixed solvent of water and organic solvent(s), or in inert organic solvent(s) such as tetrahydrofuran, benzene, toluene, xylene tetrachloromethane, chloroform, acetone or the like, or without any solvent, at −5° C. to 200° C., preferably 5° C. to 100° C., and more preferably 0° C. to 60° C.

As the amide compound, mention may be made of sodium dilauramidoglutamide lysine, sodium dimyristoylglutamide lysine, and sodium distearoylglutamide lysine.

Sodium dilauramidoglutamide lysine is in particular preferable. Sodium dilauramidoglutamide lysine is marketed as Pellicer L-10 and L-30 by Asahi Kasei Fine Chem. Col., Ltd. as an aqueous solution at a concentration of 29% by weight relative to the total weight of the aqueous solution; or as Pellicer LB-30G by Asahi Kasei Fine Chem. Co., Ltd. as a mixture of sodium dilauramidoglutamide lysine and of butyleneglycol.

In another embodiment, the (i) gemini surfactants can also comply with the following formula (I):

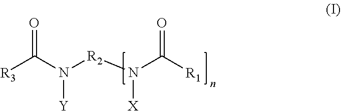

wherein:
R₁ and R₃ represent, independently of each other, an alkyl radical, comprising 1 to 25 carbon atoms;
R₂ represents a spacer group consisting of a linear or branched alkylene chain containing from 1 to 12 carbon atoms;
X and Y represent, independently of each other, a —(C₂H₄O)ₐ—(C₃H₆O)_{b}Z group, wherein:
Z represents a hydrogen atom or the radical —CH₂—COOM, —SO₃M, —P(O)(OM)₂, —C₂H₄—SO₃M, —C₃H₆—SO₃M or —CH₂(CHOH)₄CH₂OH, wherein M represent H or an alkali metal or alkali earth metal ion or an ammonium or alkanolammonium ion;
a varies from 0 to 15, b varies from 0 to 10, and the sum of a+b varies from 1 to 25; and
n varies from 1 to 10.

The gemini surfactant of formula (I) is preferably such that each one of the R₁—CO— and R₃—CO— groups comprises from 8 to 20 carbon atoms, and preferably represents a residue of coconut fatty acid (comprising mainly lauric acid and myristic acid).

In addition, this surfactant is preferably such that, for each one of the radicals X and Y, the sum of a and b has an average value that varies from 10 to 20 and is preferably equal to 15. A preferred group for Z is the —SO_{3M} group, where M is preferably an alkali metal ion such as the sodium ion.

The spacer R₂ consists advantageously of a C₁-C₃ linear alkylene chain and preferably an ethylene chain (CH₂CH₂). Finally, n is advantageously equal to 1.

A surfactant of this type is in particular the one identified by the INCI name: Sodium dicocoylethylenediamine PEG-15 sulfate, having the following structure:

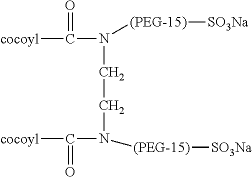

with the understanding that PEG represents the CH₂CH₂O group and that “cocoyl” represents the coconut fatty acid residue.

This surfactant has a molecular structure that is very similar to that of ceramide-3.

Preferably, the gemini surfactant according to the present invention may be used in a mixture with other surfactants, and in particular in a mixture with (a) an ester of a C₆-C₂₂ fatty acid (preferably C₁₄-C₂₀ such as stearate) and of glyceryl, (b) a diester of a C₆-C₂₂ fatty acid (preferably C₁₄-C₂₀ such as stearate) and of citric acid and glycerol (in particular a C₆-C₂₂ fatty acid ester and glyceryl monocitrate), and (c) a C₁₀-C₃₀ fatty alcohol (preferably behenyl alcohol).

Advantageously, the composition according to the present invention comprises a mixture of sodium dicocoylethylenediamine PEG-15 sulfate, of glyceryl stearate, of glyceryl stearate monocitrate and of behenyl alcohol.

For example, the gemini surfactant can be used as a mixture with other surfactants in the form of products sold by Sasol under the trade name Ceralution®, such as in particular the following products:
Ceralution® H: Behenyl Alcohol, Glyceryl Stearate, Glyceryl Stearate Citrate and Sodium Dicocoylethylenediamine PEG-15 Sulfate (INCI name),
Ceralution® F: Sodium Lauroyl Lactylate and Sodium Dicocoylethylenediamine PEG-15 Sulfate (INCI name), and
Ceralution® C: Aqua, Capric/Caprylic triglyceride, Glycerin, Ceteareth-25, Sodium Dicocoylethylenediamine PEG-15 Sulfate, Sodium Lauroyl Lactylate, Behenyl Alcohol, Glyceryl Stearate, Glyceryl Stearate Citrate, Gum Arabic, Xanthan Gum, Phenoxyethanol, Methylparaben, Ethylparaben, Butylparaben, Isobutylparaben (INCI name).

The gemini surfactant represents from 3% to 50% by weight of these mixtures. Preferably, the composition comprises, as a gemini surfactant, the compound with INCI name: Behenyl alcohol, glyceryl stearate, glyceryl stearate citrate and sodium dicocoylethylenediamine PEG-15 sulfate, sold under the trade name Ceralution® H by Sasol.

The amount of the (i) gemini surfactant(s) in the composition according to the present invention may be 0.001% by weight or more, preferably 0.005% by weight or more, and more preferably 0.01% by weight or more, relative to the total weight of the composition.

On the other hand, the amount of the (i) gemini surfactant(s) in the composition according to the present invention may be 5% by weight or less, preferably 3% by weight or less, and more preferably 1% by weight or less, relative to the total weight of the composition.

The amount of the (i) gemini surfactant(s) in the composition according to the present invention may range from 0.001% to 5% by weight, preferably from 0.005% to 3% by weight, and more preferably from 0.01% to 1% by weight, relative to the total weight of the composition.

### [Polyol]

The composition according to the present invention may further comprise at least one polyol. A single type of polyol may be used, but two or more different types of polyol may be used in combination.

The term “polyol” here means an alcohol having two or more hydroxy groups, and does not encompass a saccharide or a derivative thereof. The derivative of a saccharide includes a sugar alcohol which is obtained by reducing one or more carbonyl groups of a saccharide, as well as a saccharide or a sugar alcohol in which the hydrogen atom or atoms in one or more hydroxy groups thereof has or have been replaced with at least one substituent such as an alkyl group, a hydroxyalkyl group, an alkoxy group, an acyl group or a carbonyl group.

The polyol may be a C₂-C₁₂ polyol, preferably a C₂-C₉ polyol, comprising at least 2 hydroxy groups, and preferably 2 to 5 hydroxy groups.

The polyol may be a natural or synthetic polyol. The polyol may have a linear, branched or cyclic molecular structure.

The polyol may be selected from glycerins and derivatives thereof, and glycols and derivatives thereof. The polyol may be selected from the group consisting of glycerin, diglycerin, polyglycerin, ethyleneglycol, diethyleneglycol, propyleneglycol, dipropyleneglycol, butyleneglycol, pentyleneglycol, hexyleneglycol, 1,3-propanediol, 1,5-pentanediol, polyethyleneglycol (5 to 50 ethyleneoxide groups), and sugars such as sorbitol.

The polyol may be present in an amount ranging from 0.01% to 30% by weight, and preferably from 0.1% to 20% by weight, such as from 1% to 10% by weight, relative to the total weight of the composition.

### [Other Optional Ingredients]

The composition according to the present invention may also comprise an effective amount of other optional ingredients, known previously elsewhere in cosmetic or dermatological compositions, such as various common adjuvants, sequestering agents such as EDTA and etidronic acid, preserving agents, vitamins or provitamins, for instance, panthenol, fragrances, plant extracts, cationic polymers and so on.

The composition according to the present invention may further comprise at least one organic solvent. Thus, the organic solvent is preferably water miscible. As the organic solvent, there may be mentioned, for example, C₁-C₄ alkanols, such as ethanol and isopropanol; aromatic alcohols such as benzyl alcohol and phenoxyethanol; analogous products; and mixtures thereof.

The organic water-soluble solvents may be present in an amount ranging from 0.01% by weight or more, preferably 0.1% by weight or more, and more preferably 1% by weight or more, relative to the total weight of the composition.

The organic water-soluble solvents may be present in an amount ranging from 15% by weight or less, 50 preferably 10% by weight or less, and more preferably 5% by weight or less, relative to the total weight of the composition.

The organic water-soluble solvents may be present in an amount ranging from 0.01% to 15% by weight, preferably from 0.1% to 10% by weight, and more preferably from 1% to 5% by weight, relative to the total weight of the composition.

### [Preparation and Properties]

The composition according to the present invention can be prepared by mixing the essential ingredient(s) as explained above, and optional ingredient(s), if necessary, as explained above.

The method and means to mix the above essential and optional ingredients are not limited. Any conventional method and means can be used to mix the above essential and optional ingredients to prepare the composition according to the present invention. The conventional method and means include a homogenizer, for example a turbine mixer.

### [Form]

The composition according to the present invention is in the form of an O/W emulsion.

The composition according to the present invention comprises dispersed fatty phases dispersed in a continuous aqueous phase. The dispersed fatty phases can be in the form of oil droplets in the aqueous phase. It is preferable that the composition according to the present invention be in the form of an O/W gel emulsion or an O/W gel dispersion.

The O/W architecture or structure, which consists of fatty phases dispersed in an aqueous phase, has an external aqueous phase, and therefore, the composition according to the present invention with the O/W architecture or structure can provide a pleasant feeling during use because of the feeling of immediate freshness that the aqueous phase can provide.

### [Process and Use]

It is preferable that the composition according to the present invention be a cosmetic composition, preferably a cosmetic composition for a keratin substance such as skin, and more preferably a skin peeling composition.

The composition according to the present invention can be used for a non-therapeutic process, such as a cosmetic process, for treating a keratin substance such as skin, hair, mucous membranes, nails, eyelashes, eyebrows and/or scalp, by being applied to the keratin substance.

Thus, the present invention also relates to a cosmetic process for treating a keratin substance, comprising the step of applying the composition according to the present invention to the keratin substance.

The present invention may also relate to a use of the composition according to the present invention as a cosmetic product or in a cosmetic product such as care products, for body and/or facial skin and/or mucous membranes and/or the scalp and/or the hair and/or the nails and/or the eyelashes and/or the eyebrows.

In other words, the composition according to the present invention can be used, as it is, as a cosmetic product. Alternatively, the composition according to the present invention can be used as an element of a cosmetic product. For example the composition according to the present invention can be added to or combined with any other elements to form a cosmetic product.

The care product may be a lotion, a cream, a hair tonic, a hair conditioner, a sun screening agent, and the like.

### EXAMPLES

The present invention will be described in more detail by way of examples, which however should not be construed as limiting the scope of the present invention.

### Examples 1-2 and Comparative Examples 1-7

The following compositions according to Examples 1-2 and Comparative Examples 1-7, shown in Table 1, were prepared by mixing the components shown in Table 1 as follows:
(1) mixing the ingredients in column A in Table 1 at 70-75° C. to form a uniform mixture of Phase A;
(2) adding the ingredients in column B in Table 1 to the mixture of Phase A at 75-80° C., followed by homogenizing them to obtain a uniform mixture (Phases A and B) at 70-75° C.;
(3) mixing the ingredients in column C in Table 1 at 70-75° C. to form a mixture of Phase C;
(4) adding the mixture of Phase C to the mixture of Phases A and B at 70-75° C., followed by homogenizing them to obtain a uniform mixture (Phases A, B and C) at 70-75° C. and cooling the obtained mixture to about 30° C.; and
(5) adding the ingredient in columns D and E in Table 1 to the mixture of Phases A, B and C at about 30° C., followed by homogenizing them to obtain a uniform mixture (Phases A, B, C, D and E) at about 30° C., and cooling the obtained mixture to room temperature.

The numerical values for the amounts of the components shown in Table 1 are all based on “% by weight” as active materials.

TABLE 1

| | | | | | Comp. | Comp. | Comp. | Comp. | Comp. | Comp. | Comp. |
|---|---|---|---|---|---|---|---|---|---|---|---|
| | | Ingredient | Ex. 1 | Ex. 2 | Ex. 1 | Ex. 2 | Ex. 3 | Ex. 4 | Ex. 5 | Ex. 6 | Ex. 7 |
| C | d) | Polyglyceryl-2 Isostearate (HLB: 8) | 2 | 2 | — | — | — | 2 | 2 | 2 | 2 |
| | | Polyglyceryl-4 Caprate (HLB: 14) | 1.5 | 1.5 | — | — | 3.5 | 1.5 | 1.5 | 1.5 | 1.5 |
| | | Polyglyceryl-2 Oleate (HLB: 7) | — | — | — | 3.5 | — | — | — | — | — |
| | | Polyglyceryl-6 Distearate (HLB: 9) | 1.92 | 1.92 | — | 1.92 | 1.92 | 1.92 | 1.92 | 1.92 | 1.92 |
| | | Polyglyceryl-3 Beeswax (HLB: 4.5) | 0.255 | 0.255 | — | 0.255 | 0.255 | 0.255 | 0.255 | 0.255 | 0.255 |
| | a) | Jojoba Esters | 0.57 | 0.57 | 0.57 | 0.57 | 0.57 | — | 0.57 | 0.57 | 0.57 |
| | b) | Cetyl Alcohol | 0.255 | 0.255 | 0.255 | 0.255 | 0.255 | 0.255 | — | 0.255 | 0.255 |
| | a) | Diisopropyl Sebacate | 5 | 5 | 5 | 5 | 5 | — | 5 | 5 | 5 |
| | | Isopropyl Myristate | 2.2 | 2.2 | 2.2 | 2.2 | 2.2 | — | 2.2 | 2.2 | 2.2 |
| | | Cetyl Palmitate | 2 | 2 | 2 | 2 | 2 | — | 2 | 2 | 2 |
| | b) | Behenyl Alcohol | 1 | 1 | 1 | 1 | 1 | 1 | — | 1 | 1 |
| A | c) | Salicylic Acid | 0.198 | 0.198 | 0.198 | 0.198 | 0.198 | 0.198 | 0.198 | 0.198 | 0.198 |
| | | Phytic Acid | 0.3 | 0.3 | 0.3 | 0.3 | 0.3 | 0.3 | 0.3 | 0.3 | 0.3 |
| | | Lactic Acid | 3.33 | 3.33 | 3.33 | 3.33 | 3.33 | 3.33 | 3.33 | 3.33 | 3.33 |
| B | e) | Ammonium Acryloyldimethyltaurate/ | 0.3 | 0.3 | 0.3 | 0.3 | 0.3 | 0.3 | 0.3 | — | 0.3 |
| | | Steareth-25 Methacrylate Crosspolymer | | | | | | | | | |
| | | Xanthan Gum | 0.4 | 0.4 | 0.4 | 0.4 | 0.4 | 0.4 | 0.4 | — | 0.4 |
| D | f) | Boron Nitride | 0.4 | 0.4 | 0.4 | 0.4 | 0.4 | 0.4 | 0.4 | 0.4 | 0.4 |
| | | Calcium Aluminum Borosilicate | 1 | 1 | 1 | 1 | 1 | 1 | 1 | 1 | 1 |
| | | Titanium Oxide (and) Mica | 2 | 2 | 2 | 2 | 2 | 2 | 2 | 2 | 2 |
| A | | Sodium Dilauramidoglutamide Lysine | 0.4 | 0.4 | 0.4 | 0.4 | 0.4 | 0.4 | 0.4 | 0.4 | 0.4 |
| | g) | Water | 59.102 | 64.102 | 64.777 | 59.102 | 59.102 | 68.872 | 60.357 | 59.802 | 62.502 |
| | | Glycerin | 3 | 3 | 3 | 3 | 3 | 3 | 3 | 3 | 3 |
| | | Chlorphenesin | 0.2 | 0.2 | 0.2 | 0.2 | 0.2 | 0.2 | 0.2 | 0.2 | 0.2 |
| | | Betaine | 3 | 3 | 3 | 3 | 3 | 3 | 3 | 3 | 3 |
| | | Hydroxyacetophenone | 0.2 | 0.2 | 0.2 | 0.2 | 0.2 | 0.2 | 0.2 | 0.2 | 0.2 |
| | | Caprylyl Glycol | 0.2 | 0.2 | 0.2 | 0.2 | 0.2 | 0.2 | 0.2 | 0.2 | 0.2 |
| | | Niacinamide | 5 | — | 5 | 5 | 5 | 5 | 5 | 5 | 5 |
| | | Sodium Hydroxide | 0.66 | 0.66 | 0.66 | 0.66 | 0.66 | 0.66 | 0.66 | 0.66 | 0.66 |
| C | | Pentaerythrityl Tetra-di-t-butyl | 0.07 | 0.07 | 0.07 | 0.07 | 0.07 | 0.07 | 0.07 | 0.07 | 0.07 |
| | | Hydroxyhydrocinnamate | | | | | | | | | |
| E | | Biosaccharide Gum-2 | 0.5 | 0.5 | 0.5 | 0.5 | 0.5 | 0.5 | 0.5 | 0.5 | 0.5 |
| | | Ethanol | 3 | 3 | 3 | 3 | 3 | 3 | 3 | 3 | 3 |
| | | Fragrance | 0.04 | 0.04 | 0.04 | 0.04 | 0.04 | 0.04 | 0.04 | 0.04 | 0.04 |

| Total Amount (wt %) | 100 | 100 | 100 | 100 | 100 | 100 | 100 | 100 | 100 |
|---|---|---|---|---|---|---|---|---|---|
| Average HLB (PGFEs) | 9.8 | 9.8 | — | 9.8 | 11.9 | 9.8 | 9.8 | 9.8 | 9.8 |
| Non-Sticky Feeling | Very | Very | Good | Very | Good | Very | Poor | Good | Very |
| | Good | Good | | Good | | Poor | | | Poor |
| Smooth Feeling (Non- | Very | Very | Good | Very | Good | Very | Poor | Good | Very |
| Greasy Feeling) | Good | Good | | Good | | Poor | | | Poor |
| Temperature Stability | Very | Very | Very | Very | Poor | Very | Poor | Very | Good |
| | Good | Good | Poor | Good | | Poor | | Poor | |
| Skin Tone Improvement | Very | Fair | Very | Very | Very | Very | Very | Very | Very |
| | Good | | Good | Good | Good | Good | Good | Good | Good |

### [Evaluations]

### (Non-Sticky Feeling)

Five professional panelists evaluated “non-sticky feeling” after application of the compositions according to Examples 1-2 and Comparative Examples 1-7. Each panelist took each composition in their hands, then applied it onto their faces to evaluate non-sticky feeling after application, and graded it from 1 (poor) to 5 (very good), which was then classified in the following 4 categories based on the average of the grade:
Very Good: From 5.0 to 4.0
Good: From 3.9 to 3.0
Poor: From 2.9 to 2.0
Very Poor: From 1.9 to 1.0

The results are shown in Table 1.

### (Smooth Feeling)

Five professional panelists evaluated “smooth feeling (non-greasy feeling)” during application of the compositions according to Examples 1-2 and Comparative Examples 1-7. Each panelist took each composition in their hands, then applied it onto their faces to evaluate smooth feeling (non-greasy feeling), and graded it from 1 (poor) to 5 (very good), which was then classified in the following 4 categories based on the average of the grade:
Very Good: From 5.0 to 4.0
Good: From 3.9 to 3.0
Poor: From 2.9 to 2.0
Very Poor: From 1.9 to 1.0

The results are shown in Table 1.

### (Temperature Stability)

Each of the compositions according to Examples 1-2 and Comparative Examples 1-7 was filled into a glass bottle and was held under temperature changing conditions at 4° C., 25° C., 40° C. and 45° C. for 2 months. Each sample was then investigated for the degree of change (color, odor, and pH), and evaluated in accordance with the following criteria:
Very Good: Almost the same conditions as at production.
Good: Changes in color, odor, and pH were somewhat observed. However, no separation or no creaming or crystallizing aspect was observed.
Poor: Changes in color, odor, and pH were clearly observed. Separation, or creaming or crystallizing aspect was clearly observed.
Very Poor: Changes in color, odor, and pH were remarkably noticed. Separation, or creaming or crystallizing aspect was remarkably noticed.

The results are shown in Table 1.

### (Skin Tone Improvement)

Three professional panelists evaluated “skin tone improvement” after 4 weeks of use. Each panelist took each composition in their hands, then applied it onto their faces to evaluate skin tone improvement, and graded it from 1 (poor) to 5 (very good), which was then classified into the following 4 categories based on the average of the grade:
Very Good: From 5.0 to 4.0
Good: Less than 4.0 to 3.0
Fair: Less than 3.0 to 2.0
Poor: Less than 2.0 to 1.0

The results are shown in Table 1.

As is clear from Table 1, the compositions in the form of an O/W emulsion according to the present invention (Examples 1-2) were able to provide excellent cosmetic effects in terms of non-sticky feeling, smooth feeling, and stability under temperature changes for 2 months, which could be attributed to a combination of ingredients (a) to (g) under certain conditions. Thus, the composition according to the present invention can provide, in particular, an excellent feeling to the touch and stability for a long time period even under temperature changes.

In addition, the composition according to Example 1 which includes niacinamide was able to provide better skin tone improvement effects than the composition according to Example 2 which does not include niacinamide.

On the other hand, the composition according to Comparative Example 1 which does not include the ingredient (d) (polyglyceryl fatty acid ester having a C₆-C₃₂ fatty acid residue) showed inferior temperature stability.

The composition according to Comparative Examples 2 and 3 which do not satisfy the condition of the weighted average of HLB values of the ingredients (d) being between 8.0 and 11.5, showed inferior temperature stability.

Comparative Example 4 which does not use the ingredient (a) (oil) showed very poor cosmetic effects in terms of any of non-sticky feeling, smooth feeling and temperature stability.

Comparative Example 5 which does not use ingredient (b) (fatty alcohol) showed poor cosmetic effects in terms of any of non-sticky feeling, smooth feeling and temperature stability.

Comparative Example 6 which does not use ingredient (e) (thickener) showed very poor temperature stability.

Comparative Example 7 which does not use ingredient (f) (powder) showed very poor non-sticky feeling and smooth feeling.

1. A composition, in the form of an O/W emulsion, comprising:
(a) at least one oil;
(b) at least one fatty alcohol;
(c) at least one acid selected from hydroxyl acids, phytic acid, and mixtures thereof;
(d) at least one polyglyceryl fatty acid ester having a C₆-C₃₂ fatty acid residue;
(e) at least one thickener;
(f) at least one powder; and
(g) water,
wherein
the HLB of the (d) polyglyceryl fatty acid ester having a C₆-C₃₂ fatty acid residue or the weighted average of HLB values of the (d) polyglyceryl fatty acid esters having a C₆-C₃₂ fatty acid residue is between 8.0 and 11.5.

2. The composition according to claim 1, wherein the (a) oil is selected from the group consisting of ester oils, triglyceride oils and mixtures thereof.

3. The composition according to claim 1, wherein the amount of the (a) oil in the composition ranges from 0.01% to 30% by weight, relative to the total weight of the composition.

4. The composition according to claim 1 wherein the (b) fatty alcohol is selected from the group consisting of cetyl alcohol, cetearyl alcohol, stearyl alcohol, behenyl alcohol, oleyl alcohol, and mixtures thereof.

5. The composition according to claim 1, wherein the amount of the (b) fatty alcohol in the composition ranges from 0.01% to 15% by weight, relative to the total weight of the composition.

6. The composition according to claim 1, wherein the hydroxyl acid is selected from alpha-hydroxy acids, beta-hydroxy acids, and mixtures thereof.

7. The composition according to claim 1, wherein the amount of the (c) acid in the composition ranges from 0.01% to 15% by weight, relative to the total weight of the composition.

8. The composition according to claim 1, wherein the (d) polyglyceryl fatty acid ester having a C₆-C₃₂ fatty acid residue has a polyglyceryl moiety derived from 2 to 10 glycerins.

9. The composition according to claim 1, wherein the amount of the (d) polyglyceryl fatty acid ester having a C₆-C₃₂ fatty acid residue in the composition ranges from 0.01% to 20% by weight, relative to the total weight of the composition.

10. The composition according to claim 1, wherein the (e) thickener is selected from polysaccharides, AMPS (co)polymers, and a mixture thereof.

11. The composition according to claim 1, wherein the amount of the (e) thickener in the composition ranges from 0.01% to 10% by weight, relative to the total weight of the composition.

12. The composition according to claim 1, wherein the (f) powder is selected from the group consisting of pigments, fillers and mixtures thereof.

13. The composition according to claim 1, wherein the amount of the (f) powder(s) in the composition ranges from 0.1% to 15% by weight, relative to the total weight of the composition.

14. The composition according to claim 1, wherein the composition further comprises (h) at least one compound selected from Vitamin B3 and derivatives thereof.

15. A cosmetic process for treating a keratin substance, comprising the step of applying the composition according to claim 1 to the keratin substance.
